SEQUENCE LISTING (19 sequences)

SEQ ID NO: 1 - protein, 461 aa, homo sapiens
MNDQYHRAARDGYLELLKEATRKELNAPDEDGMTPTLWAAYHGNLESLRLIVSRGGDPDKCDIWGNTPLHLAASNGHLHCLSFLVSFGANIWCLDNDYHTPLDMAAMKGHMECVRYLDSIAAKQSSLNPKLVGKLKDKAFREAERRIRECAKLQRRHHERMERRYRRELAERSDTLSFSSLTSSTLSRRLQHLALGSHLPYSQATLHGTARGKTKMQKKLERRKQGGEGTFKVSEDGRKSARSLSGLQLGSDVMFVRQGTYANPKEWGRAPLRDMFLSDEDSVSRATLAAEPAHSEVSTDSGHDSLFTRPGLGTMVFRRNYLSSGLHGLGREDGGLDGVGAPRGRLQSSPSLDDDSLGSANSLQDRSCGEELPWDELDLGLDEDLEPETSPLETFLASLHMEDFAALLRQEKIDLEALMLCSDLDLRSISVPLGPRKKILGAVRRRRQAMERPPALEDTEL

SEQ ID NO: 2 - DNA, 3558 bp, homo sapiens
agatgtcttggtagtcgcggctctggcgctccgcaccctccgtctgcggcagcgggggctggcggccccggcccctgcccggccccgtcctccaacctcatgcctcagccctaataccgccgccctcccctgcggggggcctttctccgtgtcccccgcccgccccgtccacttcgggcgccatgaacgaccagtaccaccgggcagcccgggatggctacctggagctcctcaaggaggccacccgaaaggagctgaatgcccccgacgaggatggcatgacccccactctctgggctgcctaccatggcaacctcgagtcgctgcgtctcattgtgagccgcgggggtgacccggacaagtgtgacatctggggcaacacacccctgcatctggcagcttccaatggccacttgcactgcctgtccttcctggtgtccttcggagccaacatctggtgcctagacaacgactaccacacgccgctggacatggctgccatgaagggccacatggaatgcgtgcgctacctggactccatcgcggccaagcagagcagcctcaaccccaagctggtgggtaagctgaaggacaaggccttccgcgaggcggagcggcgcatccgcgagtgcgccaagctgcagcggaggcaccacgaacgcatggagcggcgataccggcgcgagctggccgagcgttccgacaccctcagcttctccagcctcacgtccagcaccctgagccgccggctgcagcatctggcgctgggcagccacctgccgtactctcaggccacgctgcacggcacggccaggggcaagaccaagatgcagaagaagctggagcggcgcaagcagggcggcgaaggcaccttcaaggtctccgaggatgggcgcaagagcgcccgctcgctctcgggcctgcagctgggcagcgacgtgatgttcgtgcgccagggcacctacgccaatcccaaggagtggggccgagccccgctccgggacatgttcctctcggacgaggacagcgtctcccgtgccacgctggcggccgagcctgcccactcggaggtcagcaccgactcaggccacgactccctgtttacccgccccggcctgggcaccatggtgttccgcagaaattacttgagcagtgggctgcacggactgggccgcgaggatgggggtctggatggggtgggagcgccgcggggtcggctgcagagctcccccagcctggacgatgacagcctgggcagtgccaacagcctgcaggaccgcagctgtggggaggagctgccctgggatgagctcgatttaggcttggacgaggacctggagcccgagactagcccgctggagaccttcctggcctctctgcacatggaggactttgccgccctcctgcggcaggagaagatcgacctcgaggctttgatgctgtgctctgacctcgacctccgcagcatcagcgtcccactggggccccgaaagaagatcttgggggccgtgaggaggcggcggcaggcgatggagcgcccgccggccctggaggacacagagctataaccgggggctcctctccccagaccaaaatgaattgcaagttgccacaacctatggtggggtctcgaggtcctcacagttgccagccctgcagcccccttccccaggagcaaggaccagttggggcgactcctttgaaagtctgtctgcaccttgaagctgagggtgtggcctggaggcaccagaggggcaagagaatgttccggaactccagttctgaggggtcttgaaggccctgagccacccattctgagcagccgtaaaggacatgtagacttgggggaggcctgccccagagggagggcaggggcatcggaactggatgagagtgtgggaggtggatgcgggagggtgcacctgtcccgggcgccgtggcctctcccacccctcccctgatggtcttgttctcttgtgctcaggccagagacttgctcacttcctgcattgttgtgaagggaagagtttgggctgcctcccctcctcccgtccaccccagaggggaagttcctcagccagactcttcagccaaatgccccaactttcagtctgtaccccatcccaagcatagccagctccccctccttcacctagtggtcaaggcccggagctggaaagccgggttggggtggggtgggggtactgacgcccttcccagctcatccaggggtggagcctggcctgtcttcctccaacctccccctttttcgttgtgcagcctggccccccagggaccccagagggccagggcgctagatgcaaggtgctagaccaatgggctgcagtattatagcccccaggcctggcaggcatgcgctgggctagagaaaggtgtccgccaacctcctcagtctcccaggcctgggacaggcagaacagggtgtggctgggtgtcaggcctggggtgggtaagtcctgcgatgtggacactggagaaagtggatcctctaaactgcagattgtccccaggtcaccagggctgtgtgcgtgtgcatgtttgtgtgcatgtatgcgtgtgtgcatgcatgtgtgtgtaagtatgtggtatgtgtgagcacgggtgtgtgcttgggtgtgtgtgttcttgcatacatgtgtgcgtgtgtgtgcacgtgaacgcgtgcgtgtttgtgtgtgtgcatgcatgtgtgtgcatgtgtgtgtgtgcatgcatgtgtgtgtgtgtgcatgtgtgcgtgtgtgcacactcacgttgggtggtgtttggtaggtttccctccaggtctggtagggactgggctgttgcgtgattctgcgtggatcgggggtaagagagctcacctgtgtcccagccctctgatggtggtgagaccaggggaaaggccagctcagagggtggtcacagcatgtgtcctctgacgtatgcccccaaagctctccagccagactgaggtcacagatgtgggagagtccctgatgagcccgctgacacgtgtgcgcccctttgcaccccatgcagaggtgctctcctcctttctcagccatggccccccatccctgacctcagcttgggaagcagctcctgctgggagcatggcctcagcccccagcatctcctggggcccccatggctggggcaggggaaagagagccctcaaaggcctctttctggtctgtctgggggttcccattcccacacatgtgcccagagtagggggcatggaggagatggtccctgtcctactgcaggacactgagggcttggctgcctgccaccgccatctggagcccaccacagctcttctccctgttctctgggcctgagcctggggccacccccaagtgccgacttgtttctcccagagccccaggtgctgccccttctctcctgttcctcgtccgtttactgccgcagcccctcctggttcttctggtgctggggtggagcaggctctgtgctcccaccactgtaccccgaatccctcctgcaggctcattgccacttttgtagagaatgttctctatcagtatcgta

SEQ ID NO: 3 - protein, 358 aa, homo sapiens
MAAMKGHMECVRYLDSIAAKQSSLNPKLVGKLKDKAFREAERRIRECAKLQRRHHERMERRYRRELAERSDTLSFSSLTSSTLSRRLQHLALGSHLPYSQATLHGTARGKTKMQKKLERRKQGGEGTFKVSEDGRKSARSLSGLQLGSDVMFVRQGTYANPKEWGRAPLRDMFLSDEDSVSRATLAAEPAHSEVSTDSGHDSLFTRPGLGTMVFRRNYLSSGLHGLGREDGGLDGVGAPRGRLQSSPSLDDDSLGSANSLQDRSCGEELPWDELDLGLDEDLEPETSPLETFLASLHMEDFAALLRQEKIDLEALMLCSDLDLRSISVPLGPRKKILGAVRRRRQAMERPPALEDTEL

SEQ ID NO: 4 - protein, 2215 aa, homo sapiens
MVILQQGDHVWMDLRLGQEFDVPIGAVVKLCDSGQVQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNSRDQCCIISGESGAGKTESTKLILQFLAAISGQHSWIEQQVLEATPILEAFGNAKTIRNDNSSRFGKYIDIHFNKRGAIEGAKIEQYLLEKSRVCRQALDERNYHVFYCMLEGMSEDQKKKLGLGQASDYNYLAMGNCITCEGRVDSQEYANIRSAMKVLMFTDTENWEISKLLAAILHLGNLQYEARTFENLDACEVLFSPSLATAASLLEVNPPDLMSCLTSRTLITRGETVSTPLSREQALDVRDAFVKGIYGRLFVWIVDKINAAIYKPPSQDVKNSRRSIGLLDIFGFENFAVNSFEQLCINFANEHLQQFFVRHVFKLEQEEYDLESIDWLHIEFTDNQDALDMIANKPMNIISLIDEESKFPKGTDTTMLHKLNSQHKLNANYIPPKNNHETQFGINHFAGIVYYETQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRRAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQKKSSVRHKLVHLTLKKKSKLTEEVTKRLHDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTAKELCNALADKISLKDRFGFSLYIALFDKVSSLGSGSDHVMDAISQCEQYAKEQGAQERNAPWRLFFRKEVFTPWHSPSEDNVATNLIYQQVVRGVKFGEYRCEKEDDLAELASQQYFVDYGSEMILERLLNLVPTYIPDREITPLKTLEKWAQLAIAAHKKGIYAQRRTDAQKVKEDVVSYARFKWPLLFSRFYEAYKFSGPSLPKNDVIVAVNWTGVYFVDEQEQVLLELSFPEIMAVSSSRECRVWLSLGCSDLGCAAPHSGWAGLTPAGPCSPCWSCRGAKTTAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDSVYVMPTVTMPPREIVALVTMTPDQRQDVVRLLQLRTAEPEVRAKPYTLEEFSYDYFRPPPKHTLSRVMVSKARGKDRLWSHTREPLKQALLKKLLGSEELSQEACLAFIAVLKYMGDYPSKRTRSVNELTDQIFEGPLKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIATRLLLKSSEGFSLFVKIADKVLSVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEVKQTTEPNFPEILLIAINKYGVSLIDPKTKDILTTHPFTKISNWSSGNTYFHITIGNLVRGSKLLCETSLGYKMDDLLTSYISQMLTAMSKQRGSRSGK

SEQ ID NO: 5 - protein, 899 aa, homo sapiens
MDRKVAREFRHKVDFLIENDAEKDYLYDVLRMYHQTMDVAVLVGDLKLVINEPSRLPLFDAIRPLIPLKHQVEYDQLTPRRSRKLKEVRLDRLHPEGLGLSVRGGLEFGCGLFISHLIKGGQADSVGLQVGDEIVRINGYSISSCTHEEVINLIRTKKTVSIKVRHIGLIPVKSSPDEPLTWQYVDQFVSESGGVRGSLGSPGNRENKEKKVFISLVGSRGLGCSISSGPIQKPGIFISHVKPGSLSAEVGLEIGDQIVEVNGVDFSNLDHKEAVNVLKSSRSLTISIVAAAGRELFMTDRERLAEARQRELQRQELLMQKRLAMESNKILQEQQEMERQRRKEIAQKAAEENERYRKEMEQIVEEEEKFKKQWEEDWGSKEQLLLPKTITAEVHPVPLRKPKSFGWFYRYDGKFPTIRKKGKDKKKAKYGSLQDLRKNKKELEFEQKLYKEKEEMLEKEKQLKINRLAQEVSETEREDLEESEKIQYWVERLCQTRLEQISSADNEISEMTTGPPPPPPSVSPLAPPLRRFAGGLHLHTTDLDDIPLDMFYYPPKTPSALPVMPHPPPSNPPHKVPAPPVLPLSGHVSASSSPWVQRTPPPIPIPPPPSVPTQDLTPTRPLPSALEEALSNHPFRTGDTGNPVEDWEAKNHSGKPTNSPVPEQSFPPTPKTFCPSPQPPRGPGVSTISKPVMVHQEPNFIYRPAVKSEVLPQEMLKRMVVYQTAFRQDFRKYEEGFDPYSMFTPEQIMGKDVRLLRIKKEGSLDLALEGGVDSPIGKVVVSAVYERGAAERHGGIVKGDEIMAINGKIVTDYTLAEAEAALQKAWNQGGDWIDLVVAVCPPKEYDDELASLPSSVAESPQPVRKLLEDRAAVHRHGFLLQLEPTDLLLKSKRGNQIHR

SEQ ID NO: 6 - protein, 3354 aa, homo sapiens
MGRHVATSCHVAWLLVLISGCWGQVNRLPFFTNHFFDTYLLISEDTPVGSSVTQLLAQDMDNDPLVFGVSGEEASRFFAVEPDTGVVWLRQPLDRETKSEFTVEFSVSDHQGVITRKVNIQVGDVNDNAPTFHNQPYSVRIPENTPVGTPIFIVNATDPDLGAGGSVLYSFQPPSQFFAIDSARGIVTVIRELDYETTQAYQLTVNATDQDKTRPLSTLANLAIIITDVQDMDPIFINLPYSTNIYEHSPPGTTVRIITAIDQDKGRPRGIGYTIVSGNTNSIFALDYISGVLTLNGLLDRENPLYSHGFILTVKGTELNDDRTPSDATVTTTFNILVIDINDNAPEFNSSEYSVAITELAQVGFALPLFIQVVDKDENLGLNSMFEVYLVGNNSHHFIISPTSVQGKADIRIRVAIPLDYETVDRYDFDLFANESVPDHVGYAKVKITLINENDNRPIFSQPLYNISLYENVTVGTSVLTVLATDNDAGTFGEVSYFFSDDPDRFSLDKDTGLIMLIARLDYELIQRFTLTIIARDGGGEETTGRVRINVLDVNDNVPTFQKDAYVGALRENEPSVTQLVRLRATDEDSPPNNQITYSIVSASAFGSYFDISLYEGYGVISVSRPLDYEQISNGLIYLTVMAMDAGNPPLNSTVPVTIEVFDENDNPPTFSKPAYFVSVVENIMAGATVLFLNATDLDRSREYGQESIIYSLEGSTQFRINARSGEITTTSLLDRETKSEYILIVRAVDGGVGHNQKTGIATVNITLLDINDNHPTWKDAPYYINLVEMTPPDSDVTTVVAVDPDLGENGTLVYSIQPPNKFYSLNSTTGKIRTTHAMLDRENPDPHEAELMRKIVVSVTDCGRPPLKATSSATVFVNLLDLNDNDPTFQNLPFVAEVLEGIPAGVSIYQVVAIDLDEGLNGLVSYRMPVGMPRMDFLINSSSGVVVTTTELDRERIAEYQLRVVASDAGTPTKSSTSTLTIHVLDVNDETPTFFPAVYNVSVSEDVPREFRVVWLNCTDNDVGLNAELSYFITGGNVDGKFSVGYRDAVVRTVVGLDRETTAAYMLILEAIDNGPVGKRHTGTATVFVTVLDVNDNRPIFLQSSYEASVPEDIPEGHSILQLKATDADEGEFGRVWYRILHGNHGNNFRIHVSNGLLMRGPRPLDRERNSSHVLIVEAYNHDLGPMRSSVRVIVYVEDINDEAPVFTQQQYSRLGLRETAGIGTSVIVVQATDRDSGDGGLVNYRILSGAEGKFEIDESTGLIITVNYLDYETKTSYMMNVSATDQAPPFNQGFCSVYITLLNELDEAVQFSNASYEAAILENLALGTEIVRVQAYSIDNLNQITYRFDAYTSTQAKALFKIDAITGVITVQGLVDREKGDFYTLTVVADDGGPKVDSTVKVYITVLDENDNSPRFDFTSDSAVSIPEDCPVGQRVATVKAWDPDAGSNGQVVFSLASGNIAGAFEIVTTNDSIGEVFVARPLDREELDHYILQVVASDRGTPPRKKDHILQVTILDINDNPPVIESPFGYNVSVNENVGGGTAVVQVRATDRDIGINSVLSYYITEGNKDMTFRMDRISGEIATRPAPPDRERQSFYHLVATVEDEGTPTLSATTHVYVTIVDENDNAPMFQQPHYEVLLDEGPDTLNTSLITIQALDLDEGPNGTVTYAIVAGNIVNTFRIDRHMGVITAAKELDYEISHGRYTLIVTATDQCPILSHRLTSTTTVLVNVNDINDNVPTFPRDYEGPFEVTEGQPGPRVWTFLAHDRDSGPNGQVEYSIMDGDPLGEFVISPVEGVLRVRKDVELDRETIAFYNLTICARDRGMPPLSSTMLVGIRVLDINDNDPVLLNLPMNITISENSPVSSFVAHVLASDADSGCNARLTFNITAGNRERAFFINATTGIVTVNRPLDRERIPEYKLTISVKDNPENPRIARRDYDLLLIFLSDENDNHPLFTKSTYQAEVMENSPAGTPLTVLNGPILALDADQDIYAVVTYQLLGAQSGLFDINSSTGVVTVRSGVIIDREAFSPPILELLLLAEDIGLLNSTAHLLITILDDNDNRPTFSPATLTVHLLENCPPGFSVLQVTATDEDSGLNGELVYRIEAGAQDRFLIHLVTGVIRVGNATIDREEQESYRLTVVATDRGTVPLSGTAIVTILIDDINDSRPEFLNPIQTVSVLESAEPGTVIANITAIDHDLNPKLEYHIVGIVAKDDTDRLVPNQEDAFAVNINTGSVMVKSPMNRELVATYEVTLSVIDNASDLPERSVSVPNAKLTVNVLDVNDNTPQFKPFGITYYMERILEGATPGTTLIAVAAVDPDKGLNGLVTYTLLDLVPPGYVQLEDSSAGKVIANRTVDYEEVHWLNFTVRASDNGSPPRAAEIPVYLEIVDINDNNPIFDQPSYQEAVFEDVPVGTIILTVTATDADSGNFALIEYSLGDGESKFAINPTTGDIYVLSSLDREKKDHYILTALAKDNPGDVASNRRENSVQVVIQVLDVNDCRPQFSKPQFSTSVYENEPAGTSVITMMATDQDEGPNGELTYSLEGPGVEAFHVDMDSGLVTTQRPLQSYEKFSLTVVATDGGEPPLWGTTMLLVEVIDVNDNRPVFVRPPNGTILHIREEIPLRSNVYEVYATDKDEGLNGAVRYSFLKTAGNRDWEFFIIDPISGLIQTAQRLDRESQAVYSLILVASDLGQPVPYETMQPLQVALEDIDDNEPLFVRPPKGSPQYQLLTVPEHSPRGTLVGNVTGAVDADEGPNAIVYYFIAAGNEEKNFHLQPDGCLLVLRDLDREREAIFSFIVKASSNRSWTPPRGPSPTLDLVADLTLQEVRVVLEDINDQPPRFTKAEYTAGVATDAKVGSELIQVLALDADIGNNSLVFYSILAIHYFRALANDSEDVGQVFTMGSMDGILRTFDLFMAYSPGYFVVDIVARDLAGHNDTAIIGIYILRDDQRVKIVINEIPDRVRGFEEEFIHLLSNITGAIVNTDNVQFHVDKKGRVNFAQTELLIHVVNRDTNRILDVDRVIQMIDENKEQLRNLFRNYNVLDVQPAISVRLPDDMSALQMAIIVLAILLFLAAMLFVLMNWYYRTVHKRKLKAIVAGSAGNRGFIDIMDMPNTNKYSFDGANPVWLDPFCRNLELAAQAEHEDDLPENLSEIADLWNSPTRTHGTFGREPAAVKPDDDRYLRAAIQEYDNIAKLGQIIREGPIKGSLLKVVLEDYLRLKKLFAQRMVQKASSCHSSISELIQTELDEEPGDHSPGQGSLRFRHKPPVELKGPDGIHVVHGSTGTLLATDLNSLPEEDQKGLGRSLETLTAAEATAFERNARTESAKSTPLHKLRDVIMETPLEITEL

SEQ ID NO: 7 - protein, 1955 aa, homo sapiens
MFRQFYLWTCLASGIILGSLFEICLGQYDDDCKLARGGPPATIVAIDEESRNGTILVDNMLIKGTAGGPDPTIELSLKDNVDYWVLMDPVKQMLFLNSTGRVLDRDPPMNIHSIVVQVQCINKKVGTIIYHEVRIVVRDRNDNSPTFKHESYYATVNELTPVGTTIFTGFSGDNGATDIDDGPNGQIEYVIQYNPDDPTSNDTFEIPLMLTGNIVLRKRLNYEDKTRYFVIIQANDRAQNLNERRTTTTTLTVDVLDGDDLGPMFLPCVLVPNTRDCRPLTYQAAIPELRTPEELNPIIVTPPIQAIDQDRNIQPPSDRPGILYSILVGTPEDYPRFFHMHPRTAELSLLEPVNRDFHQKFDLVIKAEQDNGHPLPAFAGLHIEILDENNQSPYFTMPSYQGYILESAPVGATISDSLNLTSPLRIVALDKDIEDTKDPELHLFLNDYTSVFTVTQTGITRYLTLLQPVDREEQQTYTFSITAFDGVQESEPVIVNIQVMDANDNTPTFPEISYDVYVYTDMRPGDSVIQLTAVDADEGSNGEITYEILVGAQGDFIINKTTGLITIAPGVEMIVGRTYALTVQAADNAPPAERRNSICTVYIEVLPPNNQSPPRFPQLMYSLEISEAMRVGAVLLNLQATDREGDSITYAIENGDPQRVFNLSETTGILTLGKALDRESTDRYILIITASDGRPDGTSTATVNIVVTDVNDNAPVFDPYLPRNLSVVEEEANAFVGQVKATDPDAGINGQVHYSLGNFNNLFRITSNGSIYTAVKLNREVRDYYELVVVATDGAVHPRHSTLTLAIKVLDIDDNSPVFTNSTYTVLVEENLPAGTTILQIEAKDVDLGANVSYRIRSPEVKHFFALHPFTGELSLLRSLDYEAFPDQEASITFLVEAFDIYGTMPPGIATVTVIVKDMNDYPPVFSKRIYKGMVAPDAVKGTPITTVYAEDADPPGLPASRVRYRVDDVQFPYPASIFEVEEDSGRVITRVNLNEEPTTIFKLVVVAFDDGEPVMSSSATVKILVLHPGEIPRFTQEEYRPPPVSELATKGTMVGVISAAAINQSIVYSIVSGNEEDTFGINNITGVIYVNGPLDYETRTSYVLRVQADSLEVVLANLRVPSKSNTAKVYIEIQDENNHPPVFQKKFYIGGVSEDARMFTSVLRVKATDKDTGNYSVMAYRLIIPPIKEGKEGFVVETYTGLIKTAMLFHNMRRSYFKFQVIATDDYGKGLSGKADVLVSVVNQLDMQVIVSNVPPTLVEKKIEDLTEILDRYVQEQIPGAKVVVESIGARRHGDAFSLEDYTKCDLTVYAIDPQTNRAIDRNELFKFLDGKLLDINKDFQPYYGEGGRILEIRTPEAVTSIKKRGESLGYTEGALLALAFIIILCCIPAILVVLVSYRQFKVRQAECTKTARIQAALPAAKPAVPAPAPVAAPPPPPPPPPGAHLYEELGDSSILFLLYHFQQSRGNNSVSEDRKHQQVVMPFSSNTIEAHKSAHVDGSLKSNKLKSARKFTFLSDEDDLSAHNPLYKENISQVSTNSDISQRTDFVDPFSPKIQAKSKSLRGPREKIQRLWSQSVSLPRRLMRKVPNRPEIIDLQQWQGTRQKAENENTGICTNKRGSSNPLLTTEEANLTEKEEIRQGETLMIEGTEQLKSLSSDSSFCFPRPHFSFSTLPTVSRTVELKSEPNVISSPAECSLELSPSRPCVLHSSLSRRETPICMLPIETERNIFENFAHPPNISPSACPLPPPPPISPPSPPPAPAPLAPPPDISPFSLFCPPPSPPSIPLPLPPPTFFPLSVSTSGPPTPPLLPPFPTPLPPPPPSIPCPPPPSASFLSTECVCITGVKCTTNLMPAEKIKSSMTQLSTTTVCKTDPQREPKGILRHVKNLAELEKSVANMYSQIEKNYLRTNVSELQTMCPSEVTNMEITSEQNKGSLNNIVEGTEKQSHSQSTSL

SEQ ID NO: 8 - protein, 187 aa, homo sapiens
MGNKQTIFTEEQLDNYQDCTFFNKKDILKLHSRFYELAPNLVPMDYRKSPIVHVPMSLIIQMPELRENPFKERIVAAFSEDGEGNLTFNDFVDMFSVLCESAPRELKANYAFKIYDFNTDNFICKEDLELTLARLTKSELDEEEVVLVCDKVIEEADLDGDGKLGFADFEDMIAKAPDFLSTFHIRI

SEQ ID NO: 9 - protein, 5202 aa, homo sapiens
MNCPVLSLGSGFLFQVIEMLIFAYFASISLTESRGLFPRLENVGAFKKVSIVPTQAVCGLPDRSTFCHSSAAAESIQFCTQRFCIQDCPYRSSHPTYTALFSAGLSSCITPDKNDLHPNAHSNSASFIFGNHKSCFSSPPSPKLMASFTLAVWLKPEQQGVMCVIEKTVDGQIVFKLTISEKETMFYYRTVNGLQPPIKVMTLGRILVKKWIHLSVQVHQTKISFFINGVEKDHTPFNARTLSGSITDFASGTVQIGQSLNGLEQFVGRMQDFRLYQVALTNREILEVFSGDLLRLHAQSHCRCPGSHPRVHPLAQRYCIPNDAGDTADNRVSRLNPEAHPLSFVNDNDVGTSWVSNVFTNITQLNQGVTISVDLENGQYQVFYIIIQFFSPQPTEIRIQRKKENSLDWEDWQYFARNCGAFGMKNNGDLEKPDSVNCLQLSNFTPYSRGNVTFSILTPGPNYRPGYNNFYNTPSLQEFVKATQIRFHFHGQYYTTETAVNLRHRYYAVDEITISGRCQCHGHADNCDTTSQPYRCLCSQESFTEGLHCDRCLPLYNDKPFRQGDQVYAFNCKPCQCNSHSKSCHYNISVDPFPFEHFRGGGGVCDDCEHNTTGRNCELCKDYFFRQVGADPSAIDVCKPCDCDTVGTRNGSILCDQIGGQCNCKRHVSGRQCNQCQNGFYNLQELDPDGCSPCNCNTSGTVDGDITCHQNSGQCKCKANVIGLRCDHCNFGFKFLRSFNDVGCEPCQCNLHGSVNKFCNPHSGQCECKKEAKGLQCDTCRENFYGLDVTNCKACDCDTAGSLPGTVCNAKTGQCICKPNVEGRQCNKCLEGNFYLRQNNSFLCLPCNCDKTGTINGSLLCNKSTGQCPCKLGVTGLRCNQCEPHRYNLTIDNFQHCQMCECDSLGTLPGTICDPISGQCLCVPNRQGRRCNQCQPGFYISPGNATGCLPCSCHTTGAVNHICNSLTGQCVCQDASIAGQRCDQCKDHYFGFDPQTGRCQPCNCHLSGALNETCHLVTGQCFCKQFVTGSKCDACVPSASHLDVNNLLGCSKTPFQQPPPRGQVQSSSAINLSWSPPDSPNAHWLTYSLLRDGFEIYTTEDQYPYSIQYFLDTDLLPYTKYSYYIETTNVHGSTRSVAVTYKTKPGVPEGNLTLSYIIPIGSDSVTLTWTTLSNQSGPIEKYILSCAPLAGGQPCVSYEGHETSATIWNLVPFAKYDFSVQACTSGGCLHSLPITVTTAQAPPQRLSPPKMQKISSTELHVEWSPPAELNGIIIRYELYMRRLRSTKETTSEESRVFQSSGWLSPHSFVESANENALKPPQTMTTITGLEPYTKYEFRVLAVNMAGSVSSAWVSERTGESAPVFMIPPSVFPLSSYSLNISWEKPADNVTRGKVVGYDINMLSEQSPQQSIPMAFSQLLHTAKSQELSYTVEGLKPYRIYEFTITLCNSVGCVTSASGAGQTLAAAPAQLRPPLVKGINSTTIHLRWFPPEELNGPSPIYQLERRESSLPALMTTMMKGIRFIGNGYCKFPSSTHPVNTDFTGIKASFRTKVPEGLIVFAASPGNQEEYFALQLKKGRLYFLFDPQGSPVEVTTTNDHGKQYSDGKWHEIIAIRHQAFGQITLDGIYTGSSAILNGSTVIGDNTGVFLGGLPRSYTILRKDPEIIQKGFVGCLKDVHFMKNYNPSAIWEPLDWQSSEEQINVYNSWEGCPASLNEGAQFLGAGFLELHPYMFHGGMNFEISFKFRTDQLNGLLLFVYNKDGPDFLAMELKSGILTFRLNTSLAFTQVDLLLGLSYCNGKWNKVIIKKEGSFISASVNGLMKHASESGDQPLVVNSPVYVGGIPQELLNSYQHLCLEQGFGGCMKDVKFTRGAVVNLASVSSGAVRVNLDGCLSTDSAVNCRGNDSILVYQGKEQSVYEGGLQPFTEYLYRVIASHEGGSVYSDWSRGRTTGAAPQSVPTPSRVRSLNGYSIEVTWDEPVVRGVIEKYILKAYSEDSTRPPRMPSASAEFVNTSNLTGILTGLLPFKNYAVTLTACTLAGCTESSHALNISTPQEAPQEVQPPVAKSLPSSLLLSWNPPKKANGIITQYCLYMDGRLIYSGSEENYIVTDLAVFTPHQFLLSACTHVGCTNSSWVLLYTAQLPPEHVDSPVLTVLDSRTIHIQWKQPRKISGILERYVLYMSNHTHDFTIWSVIYNSTELFQDHMLQYVLPGNKYLIKLGACTGGGCTVSEASEALTDEDIPEGVPAPKAHSYSPDSFNVSWTEPEYPNGVITSYGLYLDGILIHNSSELSYRAYGFAPWSLHSFRVQACTAKGCALGPLVENRTLEAPPEGTVNVFVKTQGSRKAHVRWEAPFRPNGLLTHSVLFTGIFYVDPVGNNYTLLNVTKVMYSGEETNLWVLIDGLVPFTNYTVQVNISNSQGSLITDPITIAMPPGAPDGVLPPRLSSATPTSLQVVWSTPARNNAPGSPRYQLQMRSGDSTHGFLELFSNPSASLSYEVSDLQPYTEYMFRLVASNGFGSAHSSWIPFMTAEDKPGPVVPPILLDVKSRMMLVTWQHPRKSNGVITHYNIYLHGRLYLRTPGNVTNCTVMHLHPYTAYKFQVEACTSKGCSLSPESQTVWTLPGAPEGIPSPELFSDTPTSVIISWQPPTHPNGLVENFTIERRVKGKEEVTTLVTLPRSHSMRFIDKTSALSPWTKYEYRVLMSTLHGGTNSSAWVEVTTRPSRPAGVQPPVVTVLEPDAVQVTWKPPLIQNGDILSYEIHMPDPHITLTNVTSAVLSQKVTHLIPFTNYSVTIVACSGGNGYLGGCTESLPTYVTTHPTVPQNVGPLSVIPLSESYVVISWQPPSKPNGPNLRYELLRRKIQQPLASNPPEDLNRWHNIYSGTQWLYEDKGLSRFTTYEYMLFVHNSVGFTPSREVTVTTLAGLPERGANLTASVLNHTAIDVRWAKPTVQDLQGEVEYYTLFWSSATSNDSLKILPDVNSHVIGHLKPNTEYWIFISVFNGVHSINSAGLHATTCDGEPQGMLPPEVVIINSTAVRVIWTSPSNPNGVVTEYSIYVNNKLYKTGMNVPGSFILRDLSPFTIYDIQVEVCTIYACVKSNGTQITTVEDTPSDIPTPTIRGITSRSLQIDWVSPRKPNGIILGYDLLWKTWYPCAKTQKLVQDQSDELCKAVRCQKPESICGHICYSSEAKVCCNGVLYNPKPGHRCCEEKYIPFVLNSTGVCCGGRIQEAQPNHQCCSGYYARILPGEVCCPDEQHNRVSVGIGDSCCGRMPYSTSGNQICCAGRLHDGHGQKCCGRQIVSNDLECCGGEEGVVYNRLPGMFCCGQDYVNMSDTICCSASSGESKAHIKKNDPVPVKCCETELIPKSQKCCNGVGYNPLKYVCSDKISTGMMMKETKECRILCPASMEATEHCGRCDFNFTSHICTVIRGSHNSTGKASIEEMCSSAEETIHTGSVNTYSYTDVNLKPYMTYEYRISAWNSYGRGLSKAVRARTKEDVPQGVSPPTWTKIDNLEDTIVLNWRKPIQSNGPIIYYILLRNGIERFRGTSLSFSDKEGIQPFQEYSYQLKACTVAGCATSSKVVAATTQGVPESILPPSITALSAVALHLSWSVPEKSNGVIKEYQIRQVGKGLIHTDTTDRRQHTVTGLQPYTNYSFTLTACTSAGCTSSEPFLGQTLQAAPEGVWVTPRHIIINSTTVELYWSLPEKPNGLVSQYQLSRNGNLLFLGGSEEQNFTDKNLEPNSRYTYKLEVKTGGGSSASDDYIVQTPMSTPEEIYPPYNITVIGPYSIFVAWIPPGILIPEIPVEYNVLLNDGSVTPLAFSVGHHQSTLLENLTPFTQYEIRIQACQNGSCGVSSRMFVKTPEAAPMDLNSPVLKALGSACIEIKWMPPEKPNGIIINYFIYRRPAGIEEESVLFVWSEGALEFMDEGDTLRPFTLYEYRVRACNSKGSVESLWSLTQTLEAPPQDFPAPWAQATSAHSVLLNWTKPESPNGIISHYRVVYQERPDDPTFNSPTVHAFTVKGTSHQAHLYGLEPFTTYRIGVVAANHAGEILSPWTLIQTLESSPSGLRNFIVEQKENGRALLLQWSEPMRTNGVIKTYNIFSDGFLEYSGLNRQFLFRRLDPFTLYTLTLEACTRAGCAHSAPQPLWTDEAPPDSQLAPTVHSVKSTSVELSWSEPVNPNGKIIRYEVIRRCFEGKAWGNQTIQADEKIVFTEYNTERNTFMYNDTGLQPWTQCEYKIYTWNSAGHTCSSWNVVRTLQAPPEGLSPPVISYVSMNPQKLLISWIPPEQSNGIIQSYRLQRNEMLYPFSFDPVTFNYTDEELLPFSTYSYALQACTSGGCSTSKPTSITTLEAAPSEVSPPDLWAVSATQMNVCWSPPTVQNGKITKYLVRYDNKESLAGQGLCLLVSHLQPYSQYNFSLVACTNGGCTASVSKSAWTMEALPENMDSPTLQVTGSESIEITWKPPRNPNGQIRSYELRRDGTIVYTGLETRYRDFTLTPGVEYSYTVTASNSQGGILSPLVKDRTSPSAPSGMEPPKLQARGPQEILVNWDPPVRTNGDIINYTLFIRELFERETKIIHINTTHNSFGMQSYIVNQLKPFHRYEIRIQACTTLGCASSDWTFIQTPEIAPLMQPPPHLEVQMAPGGFQPTVSLLWTGPLQPNGKVLYYELYRRQIATQPRKSNPVLIYNGSSTSFIDSELLPFTEYEYQVWAVNSAGKAPSSWTWCRTGPAPPEGLRAPTFHVISSTQAVVNISAPGKPNGIVSLYRLFSSSAHGAETVLSEGMATQQTLHGLQAFTNYSIGVEACTCFNCCSKGPTAELRTHPAPPSGLSSPQIGTLASRTASFRWSPPMFPNGVIHSYELQFHVACPPDSALPCTPSQIETKYTGLGQKASLGGLQPYTTYKLRVVAHNEVGSTASEWISFTTQKELPQYRAPFSVDSNLSVVCVNWSDTFLLNGQLKEYVLTDGGRRVYSGLDTTLYIPRTADKTFFFQVICTTDEGSVKTPLIQYDTSTGLGLVLTTPGKKKGSRSKSTEFYSELWFIVLMAMLGLILLAIFLSLILQRKIHKEPYIRERPPLVPLQKRMSPLNVYPPGENHMGLADTKIPRSGTPVSIRSNRSACVLRIPSQNQTSLTYSQGSLHRSVSQLMDIQDKKVLMDNSLWEAIMGHNSGLYVDEEDLMNAIKDFSSVTKERTTFTDTHL

SEQ ID NO: 10 - protein, 6306 aa, homo sapiens
MSVFLGPGMPSASLLVNLLSALLILFVFGETEIRFTGQTEFVVNETSTTVIRLIIERIGEPANVTAIVSLYGEDAGDFFDTYAAAFIPAGETNRTVYIAVCDDDLPEPDETFIFHLTLQKPSANVKLGWPRTVTVTILSNDNAFGIISFNMLPSIAVSEPKGRNESMPLTLIREKGTYGMVMVTFEVEGGPNPPDEDLSPVKGNITFPPGRATVIYNLTVLDDEVPENDEIFLIQLKSVEGGAEINTSRNSIEIIIKKNDSPVRFLQSIYLVPEEDHILIIPVVRGKDNNGNLIGSDEYEVSISYAVTTGNSTAHAQQNLDFIDLQPNTTVVFPPFIHESHLKFQIVDDTIPEIAESFHIMLLKDTLQGDAVLISPSVVQVTIKPNDKPYGVLSFNSVLFERTVIIDEDRISRYEEITVVRNGGTHGNVSANWVLTRNSTDPSPVTADIRPSSGVLHFAQGQMLATIPLTVVDDDLPEEAEAYLLQILPHTIRGGAEVSEPAELLFYIQDSDDVYGLITFFPMENQKIESSPGERYLSLSFTRLGGTKGDVRLLYSVLYIPAGAVDPLQAKEGILNISRRNDLIFPEQKTQVTTKLPIRNDAFLQNGAHFLVQLETVELLNIIPLIPPISPRFGEICNISLLVTPAIANGEIGFLSNLPIILHEPEDFAAEVVYIPLHRDGTDGQATVYWSLKPSGFNSKAVTPDDIGPFNGSVLFLSGQSDTTINITIKGDDIPEMNETVTLSLDRVNVENQVLKSGYTSRDLIILENDDPGGVFEFSPASRGPYVIKEGESVELHIIRSRGSLVKQFLHYRVEPRDSNEFYGNTGVLEFKPGEREIVITLLARLDGIPELDEHYWVVLSSHGERESKLGSATIVNITILKNDDPHGIIEFVSDGLIVMINESKGDAIYSAVYDVVRNRGNFGDVSVSWVVSPDFTQDVFPVQGTVVFGDQEFSKNITIYSLPDEIPEEMEEFTVILLNGTGGAKVGNRTTATLRIRRNDDPIYFAEPRVVRVQEGETANFTVLRNGSVDVTCMVQYATKDGKATARERDFIPVEKGETLIFEVGSRQQSISIFVNEDGIPETDEPFYIILLNSTGDTVVYQYGVATVIIEANDDPNGIFSLEPIDKAVEEGKTNAFWILRHRGYFGSVSVSWQLFQNDSALQPGQEFYETSGTVNFMDGEEAKPIILHAFPDKIPEFNEFYFLKLVNISGGSPGPGGQLAETNLQVTVMVPFNDDPFGVFILDPECLEREVAEDVLSEDDMSYITNFTILRQQGVFGDVQLGWEILSSEFPAGLPPMIDFLLVGIFPTTVHLQQHMRRHHSGTDALYFTGLEGAFGTVNPKYHPSRNNTIANFTFSAWVMPNANTNGFIIAKDDGNGSIYYGVKIQTNESHVTLSLHYKTLGSNATYIAKTTVMKYLEESVWLHLLIILEDGIIEFYLDGNAMPRGIKSLKGEAITDGPGILRIGAGINGNDRFTGLMQDVRSYERKLTLEEIYELHAMPAKSDLHPISGYLEFRQGETNKSFIISARDDNDEEGEELFILKLVSVYGGARISEENTTARLTIQKSDNANGLFGFTGACIPEIAEEGSTISCVVERTRGALDYVHVFYTISQIETDGINYLVDDFANASGTITFLPWQRSEVLNIYVLDDDIPELNEYFRVTLVSAIPGDGKLGSTPTSGASIDPEKETTDITIKASDHPYGLLQFSTGLPPQPKDAMTLPASSVPHITVEEEDGEIRLLVIRAQGLLGRVTAEFRTVSLTAFSPEDYQNVAGTLEFQPGERYKYIFINITDNSIPELEKSFKVELLNLEGGVAELFRVDGSGSGDGDMEFFLPTIHKRASLGVASQILVTIAASDHAHGVFEFSPESLFVSGTEPEDGYSTVTLNVIRHHGTLSPVTLHWNIDSDPDGDLAFTSGNITFEIGQTSANITVEILPDEDPELDKAFSVSVLSVSSGSLGAHINATLTVLASDDPYGIFIFSEKNRPVKVEEATQNITLSIIRLKGLMGKVLVSYATLDDMEKPPYFPPNLARATQGRDYIPASGFALFGANQSEATIAISILDDDEPERSESVFIELLNSTLVAKVQSRSIPNSPRLGPKVETIAQLIIIANDDAFGTLQLSAPIVRVAENHVGPIINVTRTGGAFADVSVKFKAVPITAIAGEDYSIASSDVVLLEGETSKAVPIYVINDIYPELEESFLVQLMNETTGGARLGALTEAVIIIEASDDPYGLFGFQITKLIVEEPEFNSVKVNLPIIRNSGTLGNVTVQWVATINGQLATGDLRVVSGNVTFAPGETIQTLLLEVLADDVPEIEEVIQVQLTDASGGGTIGLDRIANIIIPANDDPYGTVAFAQMVYRVQEPLERSSCANITVRRSGGHFGRLLLFYSTSDIDVVALAMEEGQDLLSYYESPIQGVPDPLWRTWMNVSAVGEPLYTCATLCLKEQACSAFSFFSASEGPQCFWMTSWISPAVNNSDFWTYRKNMTRVASLFSGQAVAGSDYEPVTRQWAIMQEGDEFANLTVSILPDDFPEMDESFLISLLEVHLMNISASLKNQPTIGQPNISTVVIALNGDAFGVFVIYNISPNTSEDGLFVEVQEQPQTLVELMIHRTGGSLGQVAVEWRVVGGTATEGLDFIGAGEILTFAEGETKKTVILTILDDSEPEDDESIIVSLVYTEGGSRILPSSDTVRVNILANDNVAGIVSFQTASRSVIGHEGEILQFHVIRTFPGRGNVTVNWKIIGQNLELNFANFSGQLFFPEGSLNTTLFVHLLDDNIPEEKEVYQVILYDVRTQGVPPAGIALLDAQGYAAVLTVEASDEPHGVLNFALSSRFVLLQEANITIQLFINREFGSLGAINVTYTTVPGMLSLKNQTVGNLAEPEVDFVPIIGFLILEEGETAAAINITILEDDVPELEEYFLVNLTYVGLTMAASTSFPPRLDSEGLTAQVIIDANDGARGVIEWQQSRFEVNETHGSLTLVAQRSREPLGHVSLFVYAQNLEAQVGLDYIFTPMILHFADGERYKNVNIMILDDDIPEGDEKFQLILTNPSPGLELGKNTIALIIVLANDDGPGVLSFNNSEHFFLREPTALYVQESVAVLYIVREPAQGLFGTVTVQFIVTEVNSSNESKDLTPSKGYIVLEEGVRFKALQISAILDTEPEMDEYFVCTLFNPTGGARLGVHVQTLITVLQNQAPLGLFSISAVENRATSIDIEEANRTVYLNVSRTNGIDLAVSVQWETVSETAFGMRGMDVVFSVFQSFLDESASGWCFFTLENLIYGIMLRKSSVTVYRWQGIFIPVEDLNIENPKTCEAFNIGFSPYFVITHEERNEEKPSLNSVFTFTSGFKLFLVQTIIILESSQVRYFTSDSQDYLIIASQRDDSELTQVFRWNGGSFVLHQKLPVRGVLTVALFNKGGSVFLAISQANARLNSLLFRWSGSGFINFQEVPVSGTTEVEALSSANDIYLIFAENVFLGDQNSIDIFIWEMGQSSFRYFQSVDFAAVNRIHSFTPASGIAHILLIGQDMSALYCWNSERNQFSFVLEVPSAYDVASVTVKSLNSSKNLIALVGAHSHIYELAYISSHSDFIPSSGELIFEPGEREATIAVNILDDTVPEKEESFKVQLKNPKGGAEIGINDSVTITILSNDDAYGIVAFAQNSLYKQVEEMEQDSLVTLNVERLKGTYGRITIAWEADGSISDIFPTSGVILFTEGQVLSTITLTILADNIPELSEVVIVTLTRITTEGVEDSYKGATIDQDRSKSVITTLPNDSPFGLVGWRAASVFIRVAEPKENTTTLQLQIARDKGLLGDIAIHLRAQPNFLLHVDNQATENEDYVLQETIIIMKENIKEAHAEVSILPDDLPELEEGFIVTITEVNLVNSDFSTGQPSVRRPGMEIAEIMIEENDDPRGIFMFHVTRGAGEVITAYEVPPPLNVLQVPVVRLAGSFGAVNVYWKASPDSAGLEDFKPSHGILEFADKQVTAMIEITIIDDAEFELTETFNISLISVAGGGRLGDDVVVTVVIPQNDSPFGVFGFEEKTVMIDESLSSDDPDSYVTLTVVRSPGGKGTVRLEWTIDEKAKHNLSPLNGTLHFDETESQKTIVLHTLQDTVLEEDRRFTIQLISIDEVEISPVKGSASIIIRGDKRASGEVGIAPSSRHILIGEPSAKYNGTAIISLVRGPGILGEVTVFWRIFPPSVGEFAETSGKLTMRDEQSAVIVVIQALNDDIPEEKSFYEFQLTAVSEGGVLSESSSTANITVVASDSPYGRFAFSHEQLRVSEAQRVNITIIRSSGDFGHVRLWYKTMSGTAEAGLDFVPAAGELLFEAGEMRKSLHVEILDDDYPEGPEEFSLTITKVELQGRGYDFTIQENGLQIDQPPEIGNISIVRIIIMKNDNAEGIIEFDPKYTAFEVEEDVGLIMIPVVRLHGTYGYVTADFISQSSSASPGGVDYILHGSTVTFQHGQNLSFINISIIDDNESEFEEPIEILLTGATGGAVLGRHLVSRIIIAKSDSPFGVIRFLNQSKISIANPNSTMILSLVLERTGGLLGEIQVNWETVGPNSQEALLPQNRDIADPVSGLFYFGEGEGGVRTIILTIYPHEEIEVEETFIIKLHLVKGEAKLDSRAKDVTLTIQEFGDPNGVVQFAPETLSKKTYSEPLALEGPLLITFFVRRVKGTFGEIMVYWELSSEFDITEDFLSTSGFFTIADGESEASFDVHLLPDEVPEIEEDYVIQLVSVEGGAELDLEKSITWFSVYANDDPHGVFALYSDRQSILIGQNLIRSIQINITRLAGTFGDVAVGLRISSDHKEQPIVTENAERQLVVKDGATYKVDVVPIKNQVFLSLGSNFTLQLVTVMLVGGRFYGMPTILQEAKSAVLPVSEKAANSQVGFESTAFQLMNITAGTSHVMISRRGTYGALSVAWTTGYAPGLEIPEFIVVGNMTPTLGSLSFSHGEQRKGVFLWTFPSPGWPEAFVLHLSGVQSSAPGGAQLRSGFIVAEIEPMGVFQFSTSSRNIIVSEDTQMIRLHVQRLFGFHSDLIKVSYQTTAGSAKPLEDFEPVQNGELFFQKFQTEVDFEITIINDQLSEIEEFFYINLTSVEIRGLQKFDVNWSPRLNLDFSVAVITILDNDDLAGMDISFPETTVAVAVDTTLIPVETESTTYLSTSKTTTILQPTNVVAIVTEATGVSAIPEKLVTLHGTPAVSEKPDVATVTANVSIHGTFSLGPSIVYIEEEMKNGTFNTAEVLIRRTGGFTGNVSITVKTFGERCAQMEPNALPFRGIYGISNLTWAVEEEDFEEQTLTLIFLDGERERKVSVQILDDDEPEGQEFFYVFLTNPQGGAQIVEEKDDTGFAAFAMVIITGSDLHNGIIGFSEESQSGLELREGAVMRRLHLIVTRQPNRAFEDVKVFWRVTLNKTVVVLQKDGVNLVEELQSVSGTTTCTMGQTKCFISIELKPEKVPQVEVYFFVELYEATAGAAINNSARFAQIKILESDESQSLVYFSVGSRLAVAHKKATLISLQVARDSGTGLMMSVNFSTQELRSAETIGRTIISPAISGKDFVITEGTLVFEPGQRSTVLDVILTPETGSLNSFPKRFQIVLFDPKGGARIDKVYGTANITLVSDADSQAIWGLADQLHQPVNDDILNRVLHTISMKVATENTDEQLSAMMHLIEKITTEGKIQAFSVASRTLFYEILCSLINPKRKDTRGFSHFAEVTENFAFSLLTNVTCGSPGEKSKTILDSCPYLSILALHWYPQQINGHKFEGKEGDYIRIPERLLDVQDAEIMAGKSTCKLVQFTEYSSQQWFISGNNLPTLKNKVLSLSVKGQSSQLLTNDNEVLYRIYAAEPRIIPQTSLCLLWNQAAASWLSDSQFCKVVEETADYVECACSHMSVYAVYARTDNLSSYNEAFFTSGFICISGLCLAVLSHIFCARYSMFAAKLLTHMMAASLGTQILFLASAYASPQLAEESCSAMAAVTHYLYLCQFSWMLIQSVNFWYVLVMNDEHTERRYLLFFLLSWGLPAFVVILLIVILKGIYHQSMSQIYGLIHGDLCFIPNVYAALFTAALVPLTCLVVVFVVFIHAYQVKPQWKAYDDVFRGRTNAAEIPLILYLFALISVTWLWGGLHMAYRHFWMLVLFVIFNSLQGLYVFMVYFILHNQMCCPMKASYTVEMNGHPGPSTAFFTPGSGMPPAGGEISKSTQNLIGAMEEVPPDWERASFQQGSQASPDLKPSPQNGATFPSSGGYGQGSLIADEESQEFDDLIFALKTGAGLSVSDNESGQGSQEGGTLTDSQIVELRRIPIADTHL

SEQ ID NO: 11 - protein, 907 aa, homo sapiens
MNAPLDGLSVSSSSTGSLGSAAGAGGGGGAGLRLLSANVRQLHQALTALLSEAEREQFTHCLNAYHARRNVFDLVRTLRVLLDSPVKRRLLPMLRLVIPRSDQLLFDQYTAEGLYLPATTPYRQPAWGGPDSAGPGEVRLVSLRRAKAHEGLGFSIRGGSEHGVGIYVSLVEPGSLAEKEGLRVGDQILRVNDKSLARVTHAEAVKALKGSKKLVLSVYSAGRIPGGYVTNHIYTWVDPQGRSISPPSGLPQPHGGALRQQEGDRRSTLHLLQGGDEKKVNLVLGDGRSLGLTIRGGAEYGLGIYITGVDPGSEAEGSGLKVGDQILEVNGRSFLNILHDEAVRLLKSSRHLILTVKDVGRLPHARTTVDETKWIASSRIRETMANSAGFLGDLTTEGINKPGFYKGPAGSQVTLSSLGNQTRVLLEEQARHLLNEQEHATMAYYLDEYRGGSVSVEALVMALFKLLNTHAKFSLLSEVRGTISPQDLERFDHLVLRREIESMKARQPPGPGAGDTYSMVSYSDTGSSTGSHGTSTTVSSARNTLDLEETGEAVQGNINALPDVSVDDVRSTSQGLSSFKPLPRPPPLAQGNDLPLGQPRKLGREDLQPPSSMPSCSGTVFSAPQNRSPPAGTAPTPGTSSAQDLPSSPIYASVSPANPSSKRPLDAHLALVNQHPIGPFPRVQSPPHLKSPSAEATVAGGCLLPPSPSGHPDQTGTNQHFVMVEVHRPDSEPDVNEVRALPQTRTASTLSQLSDSGQTLSEDSGVDAGEAEASAPGRGRQSVSTKSRSSKELPRNERPTDGANKPPGLLEPTSTLVRVKKSAATLGIAIEGGANTRQPLPRIVTIQRGGSAHNCGQLKVGHVILEVNGLTLRGKEHREAARIIAEAFKTKDRDYIDFLVTEFNVML

SEQ ID NO: 12 - protein, 245 aa, homo sapiens
MPSQQKKIIFCMAGVFSFACALGVVTALGTPLWIKATVLCKTGALLVNASGQELDKFMGEMQYGLFHGEGVRQCGLGARPFRFSFFPDLLKAIPVSIHVNVILFSAILIVLTMVGTAFFMYNAFGKPFETLHGPLGLYLLSFISVALWLPATRHQAQGSCGCLVMILFASEVKIHHLSEKIANYKEGTYVYKTQSEKYTTSFWVIFFCFFVHFLNGLLIRLAGFQFPFAKSKDAETTNVAADLMY

SEQ ID NO: 13 - protein, 509 aa, homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC

SEQ ID NO: 14 - DNA, 34 bp, artificial
  fwd SANS primer
gggcggccgccaccatgaatgaccagtatcaccg

SEQ ID NO: 15 - DNA, 29 bp, artificial
  rev SANS primer
ggaagcttatcatagctccgtgtcctcca

SEQ ID NO: 16 - DNA, 19 bp, artificial
  oligo fw-1
ggcctcgaagaagatcctg

SEQ ID NO: 17 - DNA, 20 bp, artificial
  oligo-rev
ggcaagtcaaaggatcagat

SEQ ID NO: 18 - DNA, 25 bp, artificial
  oligo fw-2
cagtttccccatgttgatcaccaac

SEQ ID NO: 19 - DNA, 3505 bp, homo sapiens
agatgtcttggtagtcgcggctctggcgctccgcaccctccgtctgcggcagcgggggctggcggccccggcccctgcccggccccgtcctccaacctcatgcctcagccctaataccgccgccctcccctgcggggggcctttctccgtgtcccccgcccgccccgtccacttcgggcgccatgaacgaccagtaccaccgggcagcccgggatggctacctggagctcctcaaggaggccacccgaaaggagctgaatgcccccgacgaggatggcatgacccccactctctgggctgcctaccatggcaacctcgagtcgctgcgtctcattgtgagccgcggcttccaatggccacttgcactgcctgtccttcctggtgtccttcggagccaacatctggtgcctagacaacgactaccacacgccgctggacatggctgccatgaagggccacatggaatgcgtgcgctacctggactccatcgcggccaagcagagcagcctcaaccccaagctggtgggtaagctgaaggacaaggccttccgcgaggcggagcggcgcatccgcgagtgcgccaagctgcagcggaggcaccacgaacgcatggagcggcgataccggcgcgagctggccgagcgttccgacaccctcagcttctccagcctcacgtccagcaccctgagccgccggctgcagcatctggcgctgggcagccacctgccgtactctcaggccacgctgcacggcacggccaggggcaagaccaagatgcagaagaagctggagcggcgcaagcagggcggcgaaggcaccttcaaggtctccgaggatgggcgcaagagcgcccgctcgctctcgggcctgcagctgggcagcgacgtgatgttcgtgcgccagggcacctacgccaatcccaaggagtggggccgagccccgctccgggacatgttcctctcggacgaggacagcgtctcccgtgccacgctggcggccgagcctgcccactcggaggtcagcaccgactcaggccacgactccctgtttacccgccccggcctgggcaccatggtgttccgcagaaattacttgagcagtgggctgcacggactgggccgcgaggatgggggtctggatggggtgggagcgccgcggggtcggctgcagagctcccccagcctggacgatgacagcctgggcagtgccaacagcctgcaggaccgcagctgtggggaggagctgccctgggatgagctcgatttaggcttggacgaggacctggagcccgagactagcccgctggagaccttcctggcctctctgcacatggaggactttgccgccctcctgcggcaggagaagatcgacctcgaggctttgatgctgtgctctgacctcgacctccgcagcatcagcgtcccactggggccccgaaagaagatcttgggggccgtgaggaggcggcggcaggcgatggagcgcccgccggccctggaggacacagagctataaccgggggctcctctccccagaccaaaatgaattgcaagttgccacaacctatggtggggtctcgaggtcctcacagttgccagccctgcagcccccttccccaggagcaaggaccagttggggcgactcctttgaaagtctgtctgcaccttgaagctgagggtgtggcctggaggcaccagaggggcaagagaatgttccggaactccagttctgaggggtcttgaaggccctgagccacccattctgagcagccgtaaaggacatgtagacttgggggaggcctgccccagagggagggcaggggcatcggaactggatgagagtgtgggaggtggatgcgggagggtgcacctgtcccgggcgccgtggcctctcccacccctcccctgatggtcttgttctcttgtgctcaggccagagacttgctcacttcctgcattgttgtgaagggaagagtttgggctgcctcccctcctcccgtccaccccagaggggaagttcctcagccagactcttcagccaaatgccccaactttcagtctgtaccccatcccaagcatagccagctccccctccttcacctagtggtcaaggcccggagctggaaagccgggttggggtggggtgggggtactgacgcccttcccagctcatccaggggtggagcctggcctgtcttcctccaacctccccctttttcgttgtgcagcctggccccccagggaccccagagggccagggcgctagatgcaaggtgctagaccaatgggctgcagtattatagcccccaggcctggcaggcatgcgctgggctagagaaaggtgtccgccaacctcctcagtctcccaggcctgggacaggcagaacagggtgtggctgggtgtcaggcctggggtgggtaagtcctgcgatgtggacactggagaaagtggatcctctaaactgcagattgtccccaggtcaccagggctgtgtgcgtgtgcatgtttgtgtgcatgtatgcgtgtgtgcatgcatgtgtgtgtaagtatgtggtatgtgtgagcacgggtgtgtgcttgggtgtgtgtgttcttgcatacatgtgtgcgtgtgtgtgcacgtgaacgcgtgcgtgtttgtgtgtgtgcatgcatgtgtgtgcatgtgtgtgtgtgcatgcatgtgtgtgtgtgtgcatgtgtgcgtgtgtgcacactcacgttgggtggtgtttggtaggtttccctccaggtctggtagggactgggctgttgcgtgattctgcgtggatcgggggtaagagagctcacctgtgtcccagccctctgatggtggtgagaccaggggaaaggccagctcagagggtggtcacagcatgtgtcctctgacgtatgcccccaaagctctccagccagactgaggtcacagatgtgggagagtccctgatgagcccgctgacacgtgtgcgcccctttgcaccccatgcagaggtgctctcctcctttctcagccatggccccccatccctgacctcagcttgggaagcagctcctgctgggagcatggcctcagcccccagcatctcctggggcccccatggctggggcaggggaaagagagccctcaaaggcctctttctggtctgtctgggggttcccattcccacacatgtgcccagagtagggggcatggaggagatggtccctgtcctactgcaggacactgagggcttggctgcctgccaccgccatctggagcccaccacagctcttctccctgttctctgggcctgagcctggggccacccccaagtgccgacttgtttctcccagagccccaggtgctgccccttctctcctgttcctcgtccgtttactgccgcagcccctcctggttcttctggtgctggggtggagcaggctctgtgctcccaccactgtaccccgaatccctcctgcaggctcattgccacttttgtagagaatgttctctatcagtatcgta

(19) United States
(12) Patent Application Publication
(10) Pub. No.: US 2024/0050520 A1
(43) Pub. Date: Feb. 15, 2024

(54) GENE THERAPY FOR TREATING USHER SYNDROME

(71) Applicant: INSTITUT PASTEUR, PARIS (FR)

(72) Inventors: Ghizlene LAHLOU, PARIS (FR); Saaid SAFIEDDINE, PARIS (FR); Christine PETIT, PARIS (FR)

(21) Appl. No.: 18/257,727

(22) PCT Filed: Dec. 17, 2021
(86) PCT No.: PCT/EP2021/086520

§ 371 Date: Jun. 15, 2023

(30) Foreign Application Priority Data
Dec. 18, 2020 (EP) 20306623.8

(51) Int. Cl. A61K 38/17; A61K 48/00; A61P 27/16

(52) U.S. Cl. CPC A61K 38/1709; A61K 48/0041; A61P 27/16

(57) ABSTRACT

The present invention proposes a gene therapy approach as a potential curative treatment for the USHER syndrome, in particular for the USH1G syndrome, which is characterized by a profound deafness and a severe vestibular defect in humans. More precisely, the present invention concerns a gene therapy involving administering a vector expressing a SANS protein in a time window that is compatible with human ethics and welfare i.e., in post-natal, infant and adult humans in which the auditory system is completed. The present inventors herein show for the first time that it is possible to restore genetically-impaired auditory and vestibular functions in human beings in subjects suffering from an Usher1G syndrome even when the therapeutic vector is administered at this late stage.

### SUMMARY OF THE INVENTION

The present invention proposes a gene therapy approach as a potential curative treatment for the USHER syndrome, in particular for the USH1 syndrome, which is characterized by a profound deafness and a severe vestibular defect in humans. More precisely, the present invention concerns a gene therapy involving administering a vector expressing a USHER protein in a time window that is compatible with human ethics and welfare i.e., in post-natal, infant and adult humans in which the auditory system is completed. The present inventors herein show for the first time that it is possible to restore genetically-impaired auditory and vestibular functions in human beings in subjects suffering from an Usher1G syndrome even when the therapeutic vector is administered at this late stage.

### BACKGROUND OF THE INVENTION

Deafness is the most prevalent inherited sensory disorder in humans and is a major concern and a serious burden for Public Health. Today, clinical prevalence of deafness is approximately 1 in 700 newborn. About 80% of deafness cases, associated or note with balance defect, are attributed to a genetic cause. As of now, no curative therapeutic approaches exist to cure inner ear disorders.

In particular, Usher syndrome (USH) is an autosomal recessive disease that affects both the inner ear and the retina. It is the most frequent cause of hereditary deaf-blindness, affecting 1 child in 25,000.

The following three USH clinical subtypes have been defined:
USH type I (USH1), the most severe, involves severe to profound congenital sensorineural deafness, constant vestibular dysfunction and retinitis pigmentosa with prepubertal onset;
USH type II (USH2) differs from USH1 mainly in the deafness being less severe, the absence of vestibular dysfunction and the onset of retinitis pigmentosa after puberty; and
USH type III (USH3) differing from USH1 and USH2 in the progressiveness of hearing loss and the occasional presence of vestibular dysfunction.

Seven loci responsible for the USHER syndrome type I have been defined and called USH1A-G. Five of the corresponding genes have been identified: USH1B, C, D, F and G. USH1B encodes the actin-based motor protein myosin VIIa. USH1C encodes harmonin which is a PDZ domain-containing protein. Mutations in the genes encoding two cadherin-related proteins, cadherin 23 and procadherin 15, have been shown to cause USH1D and USH1F, respectively. Studies of USH1G-affected families allowed to identify the gene SANS, the human orthologue of the gene defective in Jackson shaker (js) mutant mice, as the causative gene (Mustapha et al., 2002; Weil et al., 2003).

Current clinical approaches to remedy hearing impairment, in particular for patients suffering from the USHER syndrome, include hearing aids (for mild to moderate impairments) and cochlear implants (for severe to profound impairments). These existing solutions are however not curative treatments and are not adapted to noisy environments. Moreover, there is currently no therapeutic solution for solving the equilibrium defect and blindness displayed by severely affected USHER patients.

There is thus a need in the art for efficient therapeutic approaches to cure genetic forms of the human USHER syndrome, said therapeutic approaches being not only efficient in treating the deafness, but also the balance defects and blindness affecting these patients.

In the past, the present inventors have managed to partially restore auditory and vestibular functions in an Ush1g^{-/-} mutant knock-out mice. These mice display no identifiable auditory brainstem responses (ABR), even in response to sounds of intensities up to 110 dB SPL and present a vestibular dysfunction characterized by circling behavior and head tossing. In this prior study, the inventors had administered an AAV vector expressing the cDNA gene of the SANS gene to the immature inner ear in newborn mice. Their results showed that it was possible to completely restore the vestibular function in these mice, when administered at such an early stage of the hearing onset (WO2016/131981).

It is well-known in the art that the mouse inner ear is still structurally and functionally immature at birth, and that the hearing onset takes place in this animal species at postnatal day 12 (P12) to end around postnatal day 20 (P20) (Shnerson and Willott, J. Comp. Physiol. Psychol. 94, 36-40 (1980)). However, the hearing onset and maturation occurs in a completely different timing in humans. As a matter of fact, the human inner ear is capable of auditory function as early as 4.5 month in utero and is already ended at birth (Hepper P G & Shahidullah B S Arch Dis Child 71(2):F81-87 (1994)).

This means that the experiments of the prior art, aiming at restoring the auditory functions with vectors working before the onset of hearing or before the end of the cochlear maturation, should be performed in utero when transposed to human being trials. Nevertheless, the ultimate goal for cochlear gene therapy is the treatment of humans after a potential genetically induced deafness can be detected or diagnosed i.e., most of the time, after their birth. It is indeed difficult to develop gene therapy protocols that involve in utero delivery of treatments into the cochlea, because this surgical operation is likely to induce a number of side effects, among which a definitive hearing loss (Zhang et al, Frontiers in Molecular Neuroscience, vol. 11, Art.221, 2018).

Moreover, it is well-known that the Usher associated genetic defects are systematically diagnosed after the birth of the human babies (typically between 0 and 4 months).

To be transposable to humans, gene therapy approaches should therefore be tested and efficient in reversing established deafness phenotype affecting mature auditory systems in animal models. This will be the only way to identify treatments whose time window is compatible with human ethics and welfare, because they can be administered in post-natal, infant and/or adult humans.

It is also well known in the art that the biochemical characteristics of auditory and vestibular sensory cells (in terms of membrane receptors, signaling, etc.) dramatically change during development and maturation of the hearing and vestibular system. This explains why the efficacy and tropism of different recombinant vectors vary, depending on the developmental stage of the inner cell types of the hearing system.

In this context, the results obtained in animal models having been treated only at immature stages are difficult to translate to human. Further studies have to be carried out to bridge toward future clinical applications.

Yet, although difficult, identifying appropriate treatments that can efficiently and long lastingly restore the functionality of a high number of inner hair cells (IHC), outer hair cells (OHC) as well as vestibular hair cells (VHC) within a given therapeutic window compatible with human therapy, is essential.

In this context, the present inventors therefore sought for efficient treatments that could be used in a therapeutic window transposable to the human inner ear, i.e., that are sufficiently efficient for transfecting mature inner hair cells (IHC), outer hair cells (OHC) and vestibular hair cells (VHC) of adult mice and therefore be capable to restore their functionality in USHER suffering patients.

### DETAILED DESCRIPTION OF THE INVENTION

To solve this problem, the present inventors studied the transduction efficiency of inner ear hair cells (IHCs), outer hair cells (OHCs) and vestibular hair cells (VHCs) of several AAV vectors, at mature inner ear stages (i.e., between the 12^{th} and 30^{th} day of life of mice—P12 to P30). Recombinant vectors containing the green fluorescent protein (GFP) reporter gene driven by the ubiquitous promoter cytomegalovirus (CMV) were tested in wild-type mice. Viral preparations were microinjected directly into the cochlea of wild-type mice through the round window membrane (RWM) between P12 and P30. The cellular distribution of the GFP in the sensory epithelia of the cochlea and the vestibular end organs of the injected mice was evaluated by immunolabeling performed 1 to 2 weeks post-injection. The inventors thereby showed that several AAV serotypes can transduce the different hearing cells (IHC, OHC and VHC) at a satisfactory level, even at this late stage.

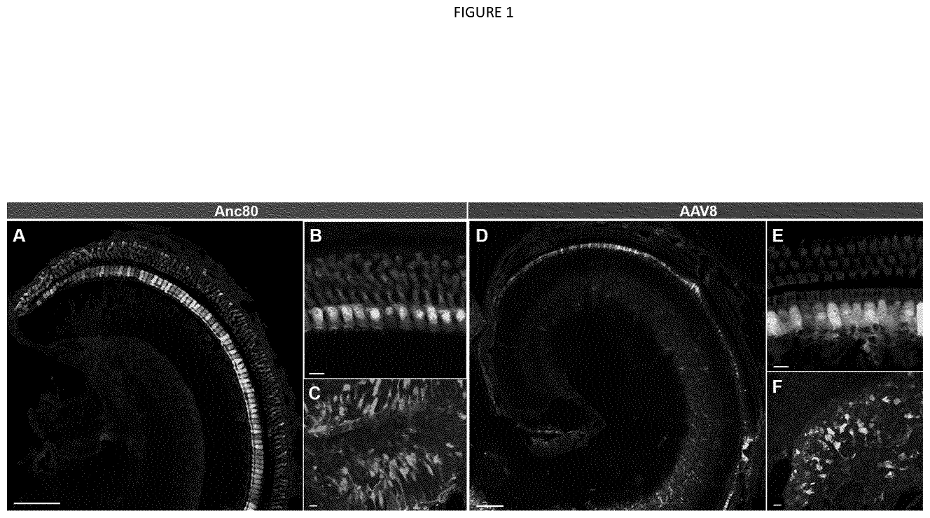

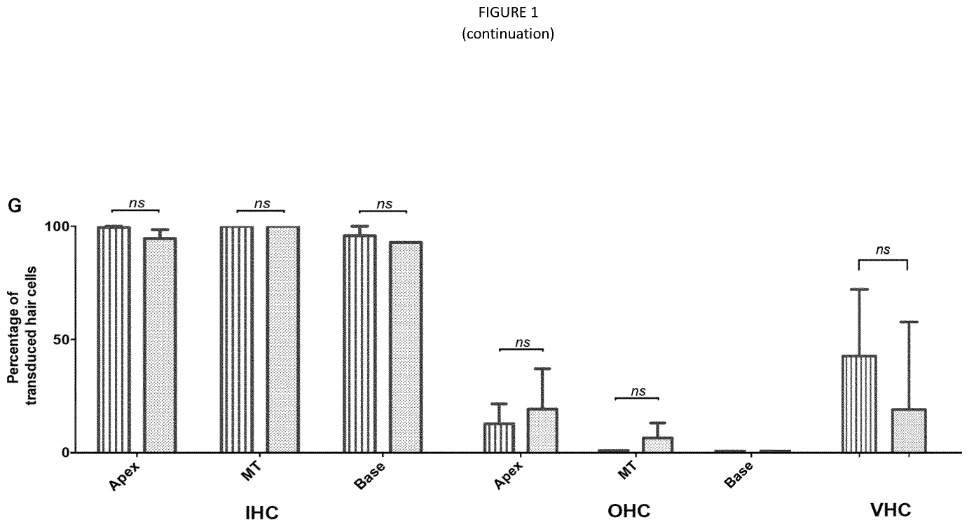

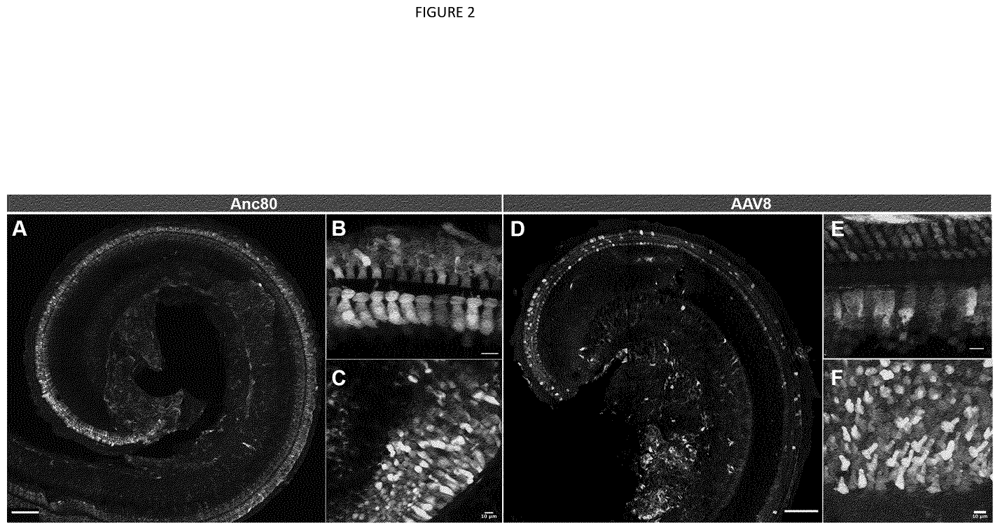

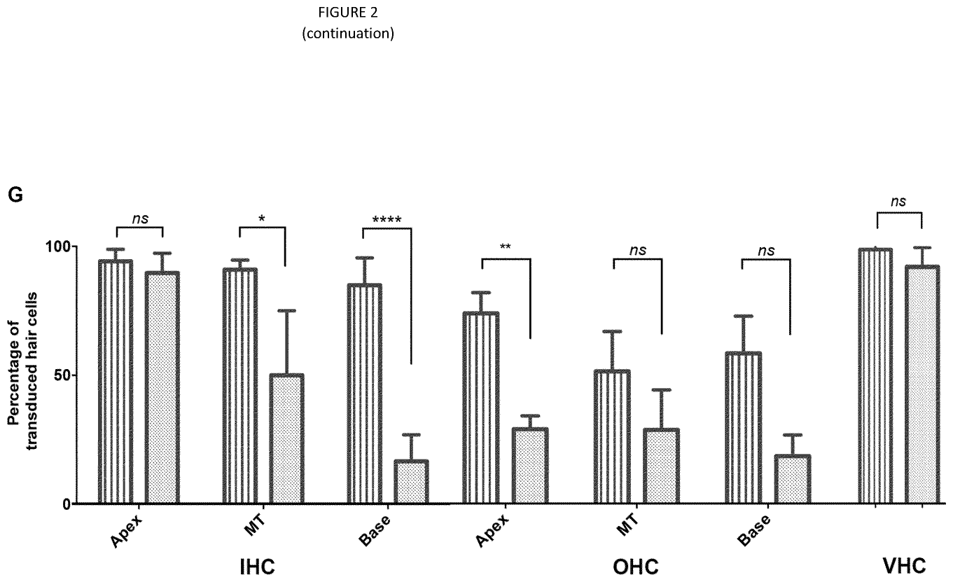

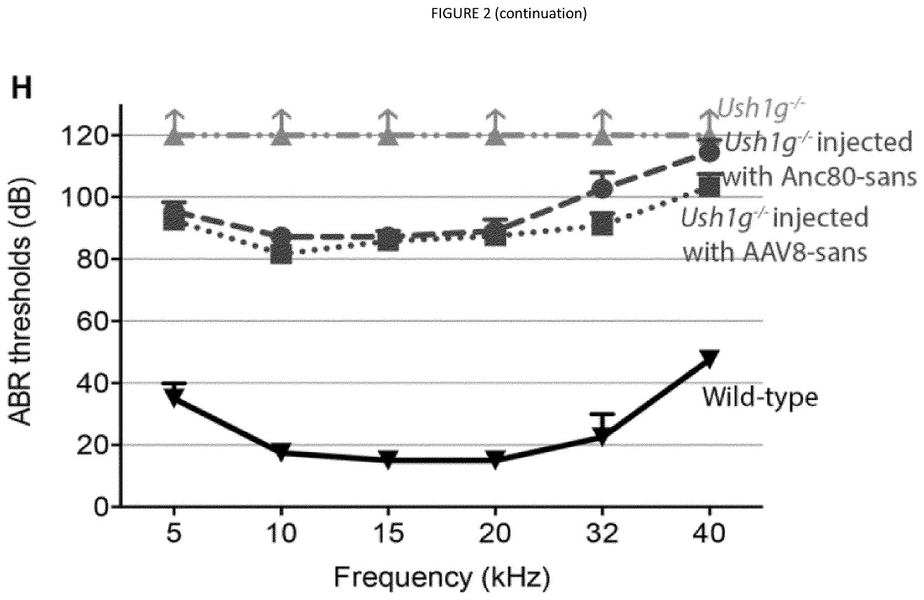

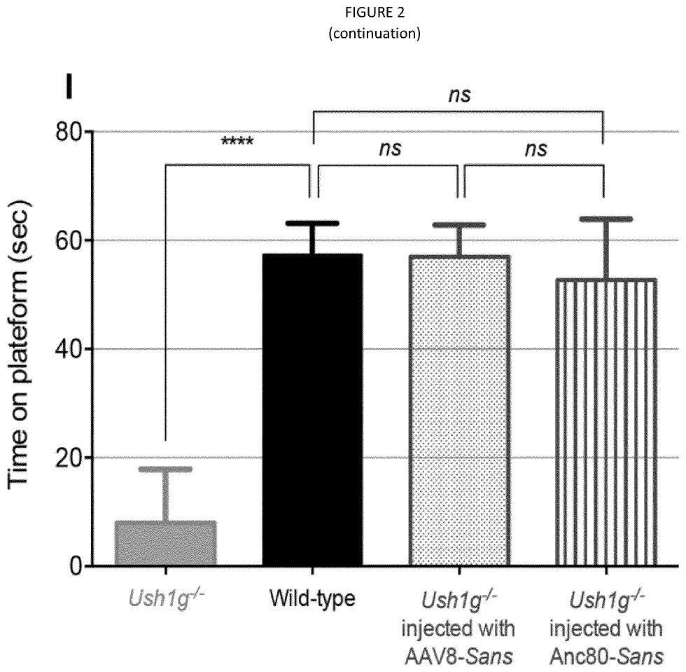

Their results furthermore showed that the transduction efficiency of the vector depends on its delivery stage: when contacted with the same vectors at P2, the IHCs, the OHCs and the VHCs were more efficiently transduced (FIG. 2G). The present inventors have however demonstrated that, despite the lower transduction rate of the vectors in OHCs and VHCs (FIG. 1G), a late administration eventually enables to restore the hearing and the vestibular functions in mice as when an early administration is done.

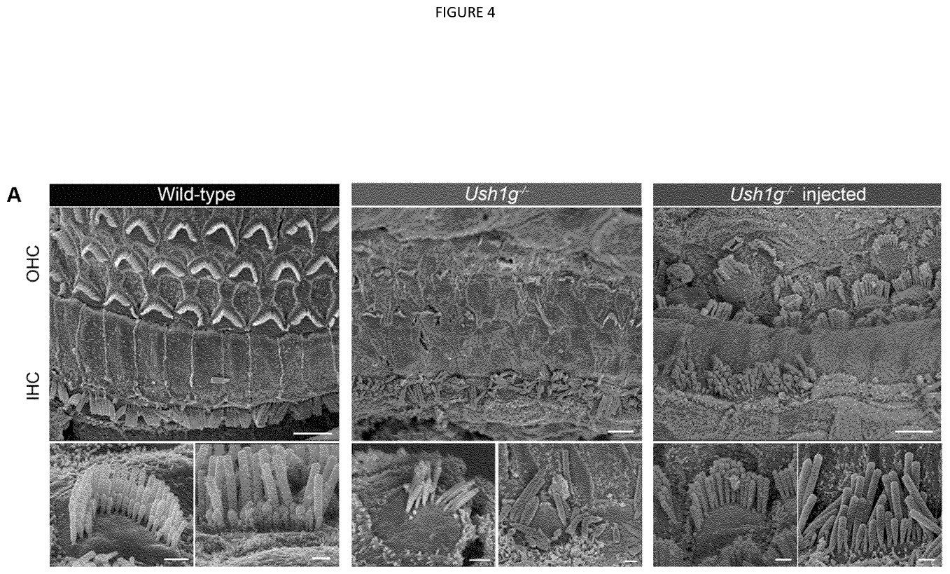

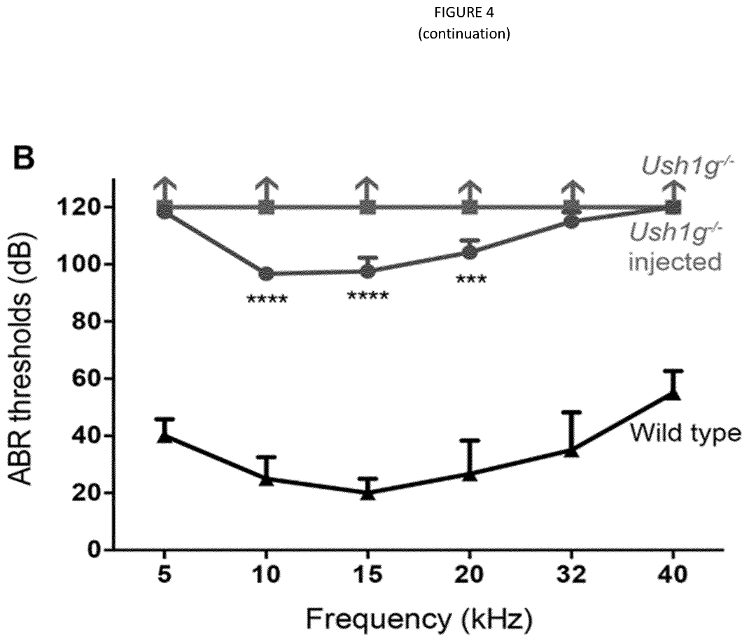

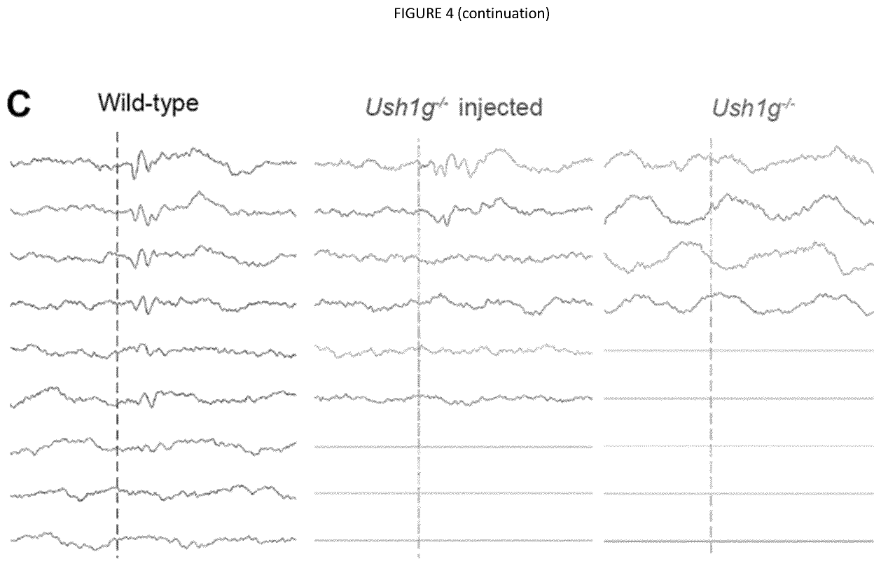

As a matter of fact, the present data strongly suggest that gene therapy carried out on USH1G animal models at adult stages with AAV vectors delivering the SANS protein can not only prevent the hair cells from degeneration, but also efficiently restore their structure and functions (FIGS. 4-7 and 9).

This is very surprising because it was known in the art that the SANS protein is a structural protein whose defect strongly affect the lifespan of the hearing cells. It was therefore thought that the absence of the SANS protein during maturation of the auditory cells in USHER patients would lead to the drastic loss of the number of hair cells, that would too irreversibly damage the auditory system (Caberlotto E. et al, PNAS 2011, Apr. 5; 108(14):5825-30).

Yet, the present results provide the first proof of concept that an equivalent clinical scenario is possible to treat patients with USH1G, months after the initial damage, when their auditory system has matured in absence of the SANS protein.

In a first aspect, the present invention relates to a vector expressing a USHER protein, or of a functional homologous or fragment thereof, for use for treating human patients suffering from the USHER syndrome, wherein said human patients have a developed and mature auditory system.

As used herein, the term “treating” is intended to mean the administration of a therapeutically effective amount of the vector of the invention to a patient suffering from the USHER syndrome, in order to restore partially or completely the hearing, the ocular and/or the vestibular function in said patient. Said recovery can be assessed by testing the auditory brain stem responses (ABRs) with electrophysiologic devices, or by testing the balance or the vision of the tested subject by appropriate means (see below). “Treatment of the USHER syndrome” is in particular intended to designate the partial or complete restoration of the hearing function and/or the balance and/or the vision of the USHER patient, regardless of the cellular mechanisms involved.

The vector of the invention can also be administered to prevent the loss of hearing, of balance or of vision induced by the worsening of the USHER syndrome. In the context of the present invention, the term “preventing” designates delaying the loss of hearing within audible frequency range, delaying the loss of vision and the unbalance symptoms in said patient.

The “Usher syndrome” or “USHER syndrome” is a rare genetic disorder primarily characterized by deafness due to an impaired ability of the inner ear and auditory nerves to transmit sensory (sound) input to the brain (sensorineural hearing loss) accompanied by retinitis pigmentosa (RP), a disorder that affects the retina and causes progressive loss of vision. RP eventually causes retinal degeneration leading to progressive blindness.

Researchers have identified three clinical types of Usher syndrome. The age at which the symptoms appear and the severity of symptoms that distinguishes the different types of Usher syndrome is determined by the underlying genetic cause. Sensorineural nerve deafness may be profound or mild, and may be progressive. The vision loss caused by RP may begin during childhood or later during life, and often first with difficulty seeing at night or in low light (“night blindness”). Issues with balance are seen in individuals with Usher syndrome types 1 and 3.

The Usher syndrome type 1 (USH1) is associated with mutations in at least the following genes: MYO7A (USH1B), USH1C, CDH23, PCDH15 (USH1F), SANS (USH1G), and possibly CIB2.

The Usher syndrome type 2 (USH2) is associated with mutations in at least the following genes: USH2A, ADGRV1 (previously called VLGR1), and USH2D.

The Usher syndrome type 3 (USH3) is associated with mutations in at least the following genes: USH3A (CLRN1) and HARS.

These genes encode proteins involved in normal hearing, vision and balance. Some of these proteins help specialized cells called hair cells to transmit sound from the inner ear to the brain and to sense light and color in the retina of the eye.

Usher syndrome affects approximately three to ten in 100,000 people worldwide. Higher than average numbers of people with Usher syndrome have been found among Jewish people in Israel, Berlin, Germany; French Canadians of Louisiana; Argentineans of Spanish descent; and Nigerian Africans. USH3, the rarest form in most populations, comprises about 40% of Usher patients in Finland. Usher syndrome is the most common genetic disorder involving both hearing and vision abnormalities. Usher syndrome types 1 and 2 account for approximately 10 percent of all cases of moderate to profound deafness in children.

The symptoms of the USHER patients are as follows:

Usher syndrome type 1 (USH1) is characterized by profound hearing loss in both ears at birth (congenital deafness) and balance problems. In many cases, affected children do not learn to walk until 18 months of age or later. Vision problems usually begin at approximately the age of ten years to early teens, although some parents report onset in children younger than 10.

Usher syndrome type 2 (USH2) is characterized by moderate to severe hearing loss in both ears at birth. In some cases, hearing loss may worsen over time. Onset of night blindness occurs during the late teens or early twenties. Peripheral vision loss is ongoing, but central vision is usually retained into adulthood. Visual problems associated with Usher syndrome type 2 tend to progress more slowly than those associated with type 1.

Usher syndrome type 3 (USH3) is characterized by later onset hearing loss, variable balance (vestibular) dysfunction and RP that can present between the second and fourth decade of life. Balance issues occur in approximately 50% of individuals with Usher syndrome type 3.

Usher syndrome is diagnosed by hearing, balance and vision examinations. A hearing (audiologic) exam measures the frequency and loudness of sounds that a person can hear. An electroretinogram measures the electrical response to the light-sensitive cells in the retina of the eyes. A retinal exam is done to observe the retina and other structures in the back of the eye. Vestibular (balance) function can be assessed by a variety of tests that evaluate different parts of the balance system. Genetic testing is clinically available for most of the genes associated with Usher syndrome.

In one aspect of the invention, the vector of the invention is administered to patients suffering from any clinical types of the Usher syndrome. By a “patient suffering from the USHER syndrome”, it is herein meant a human patient that is thought to have (or has been diagnosed to have) a mutation in any of the above-mentioned genes, leading to the diagnosis of the Usher syndrome type 1, 2 or 3.

In a particular embodiment, the vector of the invention is administered to patients suffering from the most severe form of the USHER syndrome, i.e., from the Usher syndrome type 1 (USH1), which display profound hearing impairment, early retinitis pigmentosa and constant vestibular dysfunction.

In a more particular embodiment, the vector of the invention is administered to patients having a mutation in a USH1 gene. The USH1 genes encode several proteins: Myosin VIIa (encoded by the USH1B gene), harmonin (encoded by the USH1C gene), cadherin-23 (encoded by the USH1D gene), protocadherin-15 (encoded by the USH1F gene), SANS (encoded by the USH1G gene), and CIB2 (encoded by the USH1J gene). A mutation in any of these proteins may cause the Usher syndrome type 1 to develop.

In another particular embodiment, the vector of the invention is administered to patients suffering from the USHER syndrome type 2. The USH2 genes encode several proteins: Usherin (encoded by the USH2A gene), Usher Syndrome Type-2C protein (encoded by ADGRV1 gene, previously called VLGR1), and Whirlin (encoded by the USH2D gene). A mutation in any of these proteins may cause the Usher syndrome type 2 to develop.

In another particular embodiment, the vector of the invention is administered to patients suffering from the USHER syndrome type 3. The USH3 genes encode the following proteins: Clarin-1 (encoded by the USH3A or CLRN1 gene) and Usher Syndrome 3B protein (encoded by the HARS gene). A mutation in any of these proteins may cause the Usher syndrome type 3 to develop.

Thus, the vector of the invention may contain a nucleotide sequence encoding the functional form of any of these USHER proteins, in order to prevent or treat any form of the Usher syndrome.

In the context of the present invention, the term “USHER protein” refers to any of the following proteins: Myosin VIIa (encoded by the USH1B gene), harmonin (encoded by the USH1C gene), cadherin-23 (encoded by the USH1D gene), protocadherin-15 (encoded by the USH1F gene), SANS (encoded by the USH1G gene), CIB2 (encoded by the USH1J gene), Usherin (encoded by the USH2A gene), Usher Syndrome Type-2C protein (encoded by ADGRV1 gene, previously called VLGR1), Whirlin (encoded by the USH2D gene), Clarin-1 (encoded by the USH3A or CLRN1 gene) and Usher Syndrome 3B protein (encoded by the HARS gene).

The following table summarizes the known identification numbers of these proteins:

| | | | |
|---|---|---|---|
| Protein name | Human Protein ID | Encoding gene | Human Gene ID |

USH1 proteins

| | | | |
|---|---|---|---|
| Myosin VIIa | NP_000251.3 | USH1B | 4647 |
| | (SEQ ID NO: 4) | | |
| Harmonin | NP_710142.1 | USH1C | 10083 |
| | (SEQ ID NO: 5) | | |
| Cadherin-23 | AAG27034.2 | USH1D | 64072 |
| | (SEQ ID NO: 6) | | |
| Proto-cadherin-15 | AAK31804.1 | USH1F | 65217 |
| | (SEQ ID NO: 7) | | |
| SANS | NP_775748.2 | USH1G | 124590 |
| | (SEQ ID NO: 1) | | |
| CIB2 | NP_006374.1 | USH1J | 10518 |
| | (SEQ ID NO: 8) | | |

USH2 proteins

| | | | |
|---|---|---|---|
| Usherin | NP_996816.3 | USH2A | 7399 |
| | (SEQ ID NO: 9) | | |
| Usher Syndrome Type- | NP_115495.3 | ADGRV1 | 84059 |
| 2C protein | (SEQ ID NO: 10) | | |
| Whirlin | NP_056219.3 | USH2D | 25861 |
| | (SEQ ID NO: 11) | | |

USH3 proteins

| | | | |
|---|---|---|---|
| Clarin-1 | NP_001182723 | USH3A | 7401 |
| | (SEQ ID NO: 12) | | |
| Usher Syndrome 3B | NP_002100.2 | HARS | 3035 |
| protein | (SEQ ID NO: 13) | | |

In a particular embodiment, the vector of the invention expresses a functional homologous or a fragment of said USHER protein.

In a preferred embodiment, this functional homologous polypeptide has an amino acid sequence that shares at least 70% identity and/or similarity with at least one of the USHER proteins disclosed in the above table. Said homologous polypeptide sequence more preferably shares at least 75%, and even more preferably at least 80%, or at least 90%, or at least 95%, or at least 96%, or at least 97%, or at least 98% or at least 99% identity and/or similarity with at least one of the USHER proteins disclosed in the above table. When the homologous polypeptide is much shorter than the full-length USHER protein, then local alignment can be considered. Said homologous polypeptide preferably retains at least one biological function of the USHER protein of interest.

In another embodiment, the vector of the invention allows for the expression of a functional fragment of at least one of the USHER proteins disclosed in the above table. The term “functional fragment” herein designates any fragment of the human USHER protein, or any fragment of a polypeptide having a homologous sequence as defined above, wherein said fragment retains at least one biological function of the USHER protein of interest.

The vector of the invention can be administered to patients having a mutation in any of the USH1, USH2 or USH3 genes, that are listed above. In particular, the vector of the invention can be administered to patients having a mutation in the USH1B gene, the USH1C gene, the USH1G gene, the USH2D gene, the USH2A gene, or the USH3A gene, in order to restore functional Myosin VIIa (encoded by the USH1B gene), harmonin (encoded by the USH1C gene), SANS (encoded by the USH1G gene), Whirlin (encoded by the USH2D gene), Usherin (encoded by the USH2A gene) or Clarin-1 (encoded by the USH3A gene) in said patient, respectively.

In other words, the vector of the invention preferably contains the nucleotide sequence of the USH1B gene, the USH1C gene, the USH1G gene, the USH2D gene, the USH2A gene, or the USH3A gene, and will therefore encode functional Myosin VIIa (encoded by the USH1B gene), harmonin (encoded by the USH1C gene), SANS (encoded by the USH1G gene), Whirlin (encoded by the USH2D gene), Usherin (encoded by the USH2A gene) or Clarin-1 (encoded by the USH3A gene) in the hair cells of the patients in need thereof.

In a particular embodiment, the vector of the invention is administered to patients having a mutation in a USH1 gene, in order to restore, for example, functional Myosin VIIa (encoded by the USH1B gene), harmonin (encoded by the USH1C gene), SANS (encoded by the USH1G gene), or CIB2 (encoded by the USH1J gene). In this case, the vector of the invention will contain a nucleotide sequence encoding the functional form of the said protein or a functional homologous or fragment thereof. For example, if the patient has a mutation triggering an abnormal expression and/or function of the SANS protein, then the vector of the invention will contain a polynucleotide coding for the functional form of SANS or a functional homologous or fragment thereof.

In this embodiment, the vector of the invention will contain the nucleotide sequence encoding for Myosin VIIa (the USH1B gene), harmonin (the USH1C gene), SANS (the USH1G gene), or for CIB2 (the USH1J gene) or a functional homologous or fragment thereof. Preferably, the vector of the invention contains the nucleotide sequence encoding for Myosin VIIa (the USH1B gene), harmonin (the USH1C gene), SANS (the USH1G gene), or for CIB2 (the USH1J gene) or an homologous or fragment thereof.

In a more particular embodiment, the vector of the invention is to be administered to patients having one mutation in the USH1G gene, said mutation triggering an abnormal expression, function or both, of the SANS protein.

The SANS protein plays a role in regulating endocytosis-dependent ciliogenesis (Bauss et al, 2014). It was named “SANS” because it encodes a Scaffold protein containing ANkyring repeats and a SAM (sterile alpha motif) domain. By sequence analysis, Weil et al. (2003) identified the SANS gene on chromosome 17 between markers D17S1807 and D17S1839, in human. By using biochemical and immunolocalization techniques, Adato et al. (2005) documented the interaction between SANS and harmonin, and also determined that SANS binds to myosin VIIa. The authors noted that SANS formed homomeric complex. SANS was localized to stereocilia tips of cochlear and vestibular hair cell bundle. In contrast to the other 4 known USH1 proteins, no SANS labeling was detected within the stereocilia. Adato et al. (2005) proposed that via its binding to myosin VIIa and/or harmonin, SANS controls the hair bundle cohesion and proper development by regulating the traffic of USH1 proteins en route to the stereocilia.

The SANS protein is a critical component of the tip-link complex as it is required for stereociliary elongation by controlling actin polymerization within stereocilia (Caberlotto E. et al, PNAS 2011, Apr. 5; 108(14):5825-30).

In this more particular embodiment, the vector of the invention expresses the full-length of the SANS polypeptide, or a functional homologous or fragment thereof.

As used herein, the term “SANS polypeptide” (or “SANS protein” or “ANKS4A”) designates the polypeptide encoded by the transcript variant NM_173477.5 (isoform 1, which is the longest). It has preferably the polypeptide sequence of NP_775748.2 (corresponding to the isoform 1 of the SANS protein, see also SEQ ID NO:1):

| | |
|---|---|
| 1 | mndqyhraar dgylellkea trkelnapde dgmtptlwaa yhgnleslrl ivsrggdpdk |
| 61 | cdiwgntplh laasnghlhc lsflvsfgan iwcldndyht pldmaamkgh mecvryldsi |
| 121 | aakqsslnpk lvgklkdkaf reaerrirec aklqrrhher merryrrela ersdtlsfss |
| 181 | ltsstlsrrl qhlalgshlp ysqatlhgta rgktkmqkkl errkqggegt fkvsedgrks |
| 241 | arslsglqlg sdvmfvrqgt yanpkewgra plrdmflsde dsvsratlaa epahsevstd |
| 301 | sghdslftrp glgtmvfrrn ylssglhglg redggldgvg aprgrlqssp sldddslgsa |
| 361 | nslqdrscge elpwdeldlg ldedlepets pletflaslh medfaallrq ekidlealml |
| 421 | csdldlrsis vplgprkkil gavrrrrqam erppaledte l |

In a preferred embodiment, the vector of the invention allows for the expression of a homologous polypeptide whose amino acid sequence shares at least 70% identity and/or similarity with the polypeptide sequence of NP_775748.2. Said homologous polypeptide sequence more preferably shares at least 75%, and even more preferably at least 80%, or at least 90% or at least 95%, or at least 96%, or at least 97%, or at least 98% or at least 99% identity and/or similarity with the polypeptide sequence of NP_775748.2. When the homologous polypeptide is much shorter than the polypeptide sequence of NP_775748.2 then local alignment can be considered. Said homologous polypeptide preferably retains at least one biological function of the SANS polypeptide that is of interest in the present context. As SANS is a part of the mechanotransduction machinery and essential for normal functioning of mechanotransducer channels, said biological function can be explored by recording mechanotransduction currents from inner ear hair cells using whole-cell patch clamping. This function can also be assessed by studying the aspect of hair bundles, stereocilia and hair cells in the utricle or in the cochlea of Ush1g−/− animals at a mature stage.

In a particularly preferred embodiment, the vector of the invention contains the polynucleotide of NM_173477.5. The skilled person knows that any other nucleotide sequence differing from NM_173477.5 because of the codon degeneracy can be used, as soon as it encodes the same polypeptide as NM_173477.5 does, or a homologous polypeptide as defined above.

NM_173477.5 has the following sequence (see also SEQ ID NO:2):

| | |
|---|---|
| 1 | agatgtcttg gtagtcgcgg ctctggcgct ccgcaccctc cgtctgcggc agcgggggct |
| 61 | ggcggccccg gcccctgccc ggccccgtcc tccaacctca tgcctcagcc ctaataccgc |
| 121 | cgccctcccc tgcggggggc ctttctccgt gtcccccgcc cgccccgtcc acttcgggcg |
| 181 | ccatgaacga ccagtaccac cgggcagccc gggatggcta cctggagctc ctcaaggagg |
| 241 | ccacccgaaa ggagctgaat gcccccgacg aggatggcat gacccccact ctctgggctg |
| 301 | cctaccatgg caacctcgag tcgctgcgtc tcattgtgag ccgcgggggt gacccggaca |
| 361 | agtgtgacat ctggggcaac acacccctgc atctggcagc ttccaatggc cacttgcact |
| 421 | gcctgtcctt cctggtgtcc ttcggagcca acatctggtg cctagacaac gactaccaca |
| 481 | cgccgctgga catggctgcc atgaagggcc acatggaatg cgtgcgctac ctggactcca |
| 541 | tcgcggccaa gcagagcagc ctcaacccca agctggtggg taagctgaag gacaaggcct |
| 601 | tccgcgaggc ggagcggcgc atccgcgagt gcgccaagct gcagcggagg caccacgaac |
| 661 | gcatggagcg gcgataccgg cgcgagctgg ccgagcgttc cgacaccctc agcttctcca |
| 721 | gcctcacgtc cagcaccctg agccgccggc tgcagcatct ggcgctgggc agccacctgc |
| 781 | cgtactctca ggccacgctg cacggcacgg ccaggggcaa gaccaagatg cagaagaagc |
| 841 | tggagcggcg caagcagggc ggcgaaggca ccttcaaggt ctccgaggat gggcgcaaga |
| 901 | gcgcccgctc gctctcgggc ctgcagctgg gcagcgacgt gatgttcgtg cgccagggca |
| 961 | cctacgccaa tcccaaggag tggggccgag ccccgctccg ggacatgttc ctctcggacg |
| 1021 | aggacagcgt ctcccgtgcc acgctggcgg ccgagcctgc ccactcggag gtcagcaccg |
| 1081 | actcaggcca cgactccctg tttacccgcc ccggcctggg caccatggtg ttccgcagaa |
| 1141 | attacttgag cagtgggctg cacggactgg gccgcgagga tgggggtctg gatggggtgg |
| 1201 | gagcgccgcg gggtcggctg cagagctccc ccagcctgga cgatgacagc ctgggcagtg |
| 1261 | ccaacagcct gcaggaccgc agctgtgggg aggagctgcc ctgggatgag ctcgatttag |
| 1321 | gcttggacga ggacctggag cccgagacta gcccgctgga gaccttcctg gcctctctgc |
| 1381 | acatggagga ctttgccgcc ctcctgcggc aggagaagat cgacctcgag gctttgatgc |
| 1441 | tgtgctctga cctcgacctc cgcagcatca gcgtcccact ggggccccga aagaagatct |
| 1501 | tgggggccgt gaggaggcgg cggcaggcga tggagcgccc gccggccctg gaggacacag |
| 1561 | agctataacc gggggctcct ctccccagac caaaatgaat tgcaagttgc cacaacctat |
| 1621 | ggtggggtct cgaggtcctc acagttgcca gccctgcagc ccccttcccc aggagcaagg |
| 1681 | accagttggg gcgactcctt tgaaagtctg tctgcacctt gaagctgagg gtgtggcctg |
| 1741 | gaggcaccag aggggcaaga gaatgttccg gaactccagt tctgaggggt cttgaaggcc |
| 1801 | ctgagccacc cattctgagc agccgtaaag gacatgtaga cttgggggag gcctgcccca |
| 1861 | gagggagggc aggggcatcg gaactggatg agagtgtggg aggtggatgc gggagggtgc |
| 1921 | acctgtcccg ggcgccgtgg cctctcccac ccctcccctg atggtcttgt tctcttgtgc |
| 1981 | tcaggccaga gacttgctca cttcctgcat tgttgtgaag ggaagagttt gggctgcctc |
| 2041 | ccctcctccc gtccacccca gaggggaagt tcctcagcca gactcttcag ccaaatgccc |
| 2101 | caactttcag tctgtacccc atcccaagca tagccagctc cccctccttc acctagtggt |
| 2161 | caaggcccgg agctggaaag ccgggttggg gtggggtggg ggtactgacg cccttcccag |
| 2221 | ctcatccagg ggtggagcct ggcctgtctt cctccaacct cccccttttt cgttgtgcag |
| 2281 | cctggccccc cagggacccc agagggccag ggcgctagat gcaaggtgct agaccaatgg |
| 2341 | gctgcagtat tatagccccc aggcctggca ggcatgcgct gggctagaga aaggtgtccg |
| 2401 | ccaacctcct cagtctccca ggcctgggac aggcagaaca gggtgtggct gggtgtcagg |
| 2461 | cctggggtgg gtaagtcctg cgatgtggac actggagaaa gtggatcctc taaactgcag |
| 2521 | attgtcccca ggtcaccagg gctgtgtgcg tgtgcatgtt tgtgtgcatg tatgcgtgtg |
| 2581 | tgcatgcatg tgtgtgtaag tatgtggtat gtgtgagcac gggtgtgtgc ttgggtgtgt |
| 2641 | gtgttcttgc atacatgtgt gcgtgtgtgt gcacgtgaac gcgtgcgtgt ttgtgtgtgt |
| 2701 | gcatgcatgt gtgtgcatgt gtgtgtgtgc atgcatgtgt gtgtgtgtgc atgtgtgcgt |
| 2761 | gtgtgcacac tcacgttggg tggtgtttgg taggtttccc tccaggtctg gtagggactg |
| 2821 | ggctgttgcg tgattctgcg tggatcgggg gtaagagagc tcacctgtgt cccagccctc |
| 2881 | tgatggtggt gagaccaggg gaaaggccag ctcagagggt ggtcacagca tgtgtcctct |
| 2941 | gacgtatgcc cccaaagctc tccagccaga ctgaggtcac agatgtggga gagtccctga |
| 3001 | tgagcccgct gacacgtgtg cgcccctttg caccccatgc agaggtgctc tcctcctttc |
| 3061 | tcagccatgg ccccccatcc ctgacctcag cttgggaagc agctcctgct gggagcatgg |
| 3121 | cctcagcccc cagcatctcc tggggccccc atggctgggg caggggaaag agagccctca |
| 3181 | aaggcctctt tctggtctgt ctgggggttc ccattcccac acatgtgccc agagtagggg |
| 3241 | gcatggagga gatggtccct gtcctactgc aggacactga gggcttggct gcctgccacc |
| 3301 | gccatctgga gcccaccaca gctcttctcc ctgttctctg ggcctgagcc tggggccacc |
| 3361 | cccaagtgcc gacttgtttc tcccagagcc ccaggtgctg ccccttctct cctgttcctc |
| 3421 | gtccgtttac tgccgcagcc cctcctggtt cttctggtgc tggggtggag caggctctgt |
| 3481 | gctcccacca ctgtaccccg aatccctcct gcaggctcat tgccactttt gtagagaatg |
| 3541 | ttctctatca gtatcgta |

In the context of the invention, when the identity percentage between said two homologous sequences can be identified by a global alignment of the sequences in their entirety (e.g., when the sequences are of about the same size), this alignment can be performed by means of an algorithm that is well known by the skilled person, such as the one disclosed in Needleman and Wunsch (1970). Accordingly, sequence comparisons between two amino acid sequences or two nucleotide sequences can be performed for example by using any software known by the skilled person, such as the “needle” software using the “Gap open” parameter of 10, the “Gap extend” parameter of 0.5 and the “Blosum 62” matrix.

When local alignment of the sequences is to be considered (e.g., in case of homologs that have a smaller size than the sequences of the invention), then said alignment can be performed by means of a conventional algorithm such as the one disclosed in Smith and Waterman (J. Mol. Evol. 1981; 18(1) 38-46).

The invention provides systems encoding homologous amino acid sequences that are “similar” to each other. “Similarity” of two targeted amino acid sequences can be determined by calculating a similarity score for the two amino acid sequences. As used herein, the “similarity score” refers to the score generated for the two sequences using the BLOSUM62 amino acid substitution matrix, a gap existence penalty of 11, and a gap extension penalty of 1, when the two sequences are optimally aligned. Two sequences are “optimally aligned” when they are aligned so as to produce the maximum possible score for that pair of sequences, which might require the introduction of gaps in one or both of the sequences to achieve that maximum score. Two amino acid sequences are substantially similar if their similarity score exceeds a certain threshold value. The threshold value can be any integer ranging from at least 1190 to the highest possible score for a particular reference sequence. For example, the threshold similarity score can be 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, or higher. If in a particular embodiment of the invention, the threshold score is set at, for example, 1300, then any amino acid sequence that can be optimally aligned with a reference sequence to generate a similarity score of greater than 1300 is “similar” to said reference sequence. Amino acid substitution matrices and their use in quantifying the similarity between two sequences are well-known in the art and described, e.g., in Dayhoff et al. (1978), “A model of evolutionary change in proteins”, “Atlas of Protein Sequence and Structure,” Vol. 5, Suppl. 3 (ed. M. O. Dayhoff), pp. 345-352. Natl. Biomed. Res. Found., Washington, D.C. and in Henikoff et al. (1992) Proc. Natl. Acad. Sci. USA 89:10915-10919. While optimal alignment and scoring can be accomplished manually, the process is facilitated by the use of a computer-implemented alignment algorithm, e.g., gapped BLAST 2.0, described in Altschul et al., (1997) Nucleic Acids Res. 25:3389-3402, and made available to the public at the National Center for Biotechnology Information website. To generate accurate similarity scores using NCBI BLAST, it is important to turn off any filtering, e.g., low complexity filtering, and to disable the use of composition based statistics. One should also confirm that the correct substitution matrix and gap penalties are used. Optimal alignments, including multiple alignments, can be prepared using, e.g., PSI-BLAST, available through the NCBI internet site and described by Altschul et al., (1997) Nucleic Acids Res. 25:3389-3402.

The functional SANS protein is composed of three domains: the N-terminal (N-term) domain contains three ankyrin-repeats, the central domain (CENT) and a C-terminal domain which includes a SAM (sterile alpha motif) followed by a class I PBM (PDZ-binding motif). The CENT domain has been identified as the major interaction domain in the SANS molecule. Yeast-2-hybrid screens and binary protein-protein interaction assays revealed more than 50 putative and validated binding proteins which bind to the CENT domain (Sorusch et al, 2019).

In another embodiment, the vector of the invention allows for the expression of a functional fragment of the SANS polypeptide. The term “functional fragment” herein designates any fragment of the human SANS polypeptide or any fragment of a polypeptide having a homologous sequence as defined above, wherein said fragment retains at least one biological function of the SANS polypeptide that is of interest in the present context. As SANS is a part of the mechanotransduction machinery and essential for normal functioning of mechanotransducer channels, this biological function can be explored by recording mechanotransduction currents from inner ear hair cells using whole-cell patch clamping. This function can also be assessed by studying the aspect of hair bundles, stereocilia and hair cells in the utricle or in the cochlea of Ush1g^{-/-} animals at a mature stage.

For example, said functional fragment can have the amino acid sequence presented in NP_001269418.1 (SEQ ID NO:3). Said sequence characterises the isoform 2 of the wild-type human SANS polypeptide. This isoform 2 is encoded by the cDNA variant having the sequence NM_001282489.3 (SEQ ID NO:19). This variant differs in its 5′-UTR and initiates translation at a downstream start codon, compared to isoform 1 of SANS.

A number of viral and nonviral vectors have been developed for delivery of genetic material in various tissues and organs. In most cases, these vectors are replication incompetent and pose little threat of viral-induced disease. Rather, the viral genome has been partly or fully deleted, expanding the capacity to allow inclusion of therapeutic DNA cargo within the viral capsid. Some vectors include single-stranded DNA, while others include double-stranded DNA. Particularly preferred vectors in the context of the invention are lentiviral vectors, adenovirus vectors, Adeno-associated viruses (AAV) as disclosed in Ahmed et al, JARO 18:649-670 (2017).

Specifically, AAVs are small replication-deficient adenovirus-dependent viruses from the Parvoviridae family. They have an icosaedrical capsid of 20-25 nm in diameter and a genome of 4.8 kb flanked by two inverted terminal repeats (ITRs). After uncoating in a host cell, the AAV genome can persist in a stable episome state by forming high molecular weight head-to-tail circular concatamers, or can integrate into the host cell genome. Both scenarios provide long-term and high-level transgene expression.

AAV appears to be a promising virus for cochlear gene therapies based on results obtained in human trials of ocular gene therapy. The reasons for the success of AAV in human ocular gene therapy include: (1) proven safety profile (large number of human trials have shown that AAV lack pathogenicity and possess very low immunogenicity), (2) long-lasting transgene expression in non-dividing cells, (3) the small size of AAV (≈20 nm, which is five times smaller than Adenoviruses) helps the diffusion across cellular barriers to reach targeted cells (Zhang et al, Frontiers in Molecular Neuroscience, vol. 11, Art.221, 2018).

Twelve natural occurring serotypes of human AAV have been characterized to date. They have distinct transduction specificities or tropisms making them valuable tools to direct transgene expression to a specific subset of cochlear cells. In addition, the small genome of the AAV can be easily manipulated, which increases the versatility of the AAV production system, thereby easing the generation of hybrid AAV by cross-packaging assay of different serotypes containing the same inverted terminal repeat (ITR) sequences from the AAV of serotype 2 (AAV2, the first serotype isolated and historically adopted as a gene therapy vector). The same transgene, flanked by the AAV2 ITRs and any of the available AAV capsids, can thus be used for various therapeutic application. AAVs obtained by this process are named depending on the original source of their ITRs and capsid. For example, AAV2/8 is a hybrid vector containing the ITR of AAV2 pseudotyped with the AAV8 capsid. Many of these serotypes have inherent tropisms and transduction efficiencies in muscles, lung, liver, brain, retina, and vasculature. Multiple attempts of AAV pseudotyping and capsid engineering resulted in considerable improvement of tropism and efficiency of transduction. As for cells of the inner ear, AAV1-4, 7, and 8 were shown to infect spiral limbus, spiral ligament, and spiral ganglion cells in vivo. Infection of IHCs was also shown for AAV1-3, 5, 6, and 8. AAV1 was the most effective and occasionally infected OHCs and supporting cells. Also, AAV5 was shown to be efficient for Claudius cells, spiral ganglion, and inner sulcus cells. Among pseudotyped vectors, AAV2/1 was found to efficiently transduce progenitor cells giving rise to IHCs and OHCs in mouse cochlea, and AAV2/2 was optimal for IHCs of guinea pig cochlea (Ahmed et al, JARO 18:649-670 (2017)).

Thus, in a preferred embodiment, the vector of the invention contains an AAV vector chosen in the group consisting of: AAV1, AAV2, AAV3, AAV4, AAV5, AAV6, AAV7, AAV8, AAV9, and AAV10.

In a more preferred embodiment, the serotype of the vector of the invention is AAV2, AAV8, AAV5, AAV9 or AAV1. In an even more preferred embodiment, the serotype of said vector is AAV2. In another more preferred embodiment, the serotype of said vector is AAV8.

In another more preferred embodiment, the vector of the invention contains the ITR of AAV2 pseudotyped with the AAV8 capsid.

In this particular aspect, the present invention relates to the use of AAV2/8 vectors expressing at least one USHER protein as defined above, or a functional homologous or fragment thereof, preferably the SANS gene product or a functional homologous or fragment thereof, for treating human patients suffering from the USHER syndrome, wherein said human patients have a developed and mature auditory system.

In order to increase the efficacy of gene expression, and prevent the unintended spread of the virus, genetic modifications of AAV can be performed. These genetic modifications include the deletion of the E1 region, deletion of the E1 region along with deletion of either the E2 or E4 region, or deletion of the entire adenovirus genome except the cis-acting inverted terminal repeats and a packaging signal. Such vectors are advantageously encompassed in the present invention.

Moreover, genetically modified AAV having a mutated capsid protein may be used so as to direct the gene expression towards a particular tissue type, e.g., to auditory cells. In this aim, modified serotype-2 and -8 AAV vectors in which tyrosine residues in the viral envelope are substituted for alanine residues can be used. In the case of tyrosine mutant serotype-2, tyrosine 444 can be substituted with alanine (AAV2-Y444A). In the case of serotype 8, tyrosine 733 can be substituted with an alanine reside (AAV8-Y733A). By using AAV2-Y444A or AAV8-Y733A, it is possible to increase gene transfer by up to 10,000 fold, decreasing the amount of AAV necessary to infect the sensory hair cells of the cochlea.

In a preferred embodiment, the polynucleotide(s) of the invention expressing the USHER polypeptide or gene or functional fragment thereof, is contained in recombinant AAV2 particles in which all the tyrosine residues have been replaced by phenylalanine residues (AAV2 (Y->F) or Quad Y-F, as disclosed in Petrs-Silva H et al, Mol. Ther. 19, 293-301 (2011) and in the examples below. Mutated tyrosine residues on the outer surface of the capsid proteins include, for example, but are not limited to, mutations of Tyr252 to Phe252 (Y252F), Tyr272 to Phe272 (Y272F), Tyr444 to Phe444 (Y444F), Tyr500 to Phe500 (Y500F), Tyr700 to Phe700 (Y700F), Tyr704 to Phe704 (Y704F), Tyr730 to Phe730 (Y730F) and Tyr733 to Phe733 (Y733F). These modified vectors facilitate penetration of the vector across the round window membranes, which allow for non-invasive delivery of the vectors to the hair cells/spiral ganglion neurons of the cochlea. These mutated vectors avoid degradation by the proteasome, and their transduction efficiency is significantly increased.

Other recombinant AAV particles that derivate from the natural serotypes 1-10 include AAV2-AAV3 hybrids, AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6 (Y445F/Y731F), AAV2.5T, AAV-HAE1/2, AAV clone 32/83, AAVShH10, AAV8 (Y733F), AAV2.15, AAV2.4, AAVM41, and AAVr3.45 (Asokan A. et al, Mol. Therapy, vol. 20 no 4, 699-708, 2012).

In a particular aspect, the present invention relates to the use of the synthetic vector Anc80L65 expressing at least one USHER protein or a functional homologous or fragment thereof, preferably the SANS gene product or a functional homologous or fragment thereof, for treating human patients suffering from the USHER syndrome, wherein said human patients have a developed and mature auditory system.

It is also possible to use exosome-associated AAVs as proposed by György et al, Mol. Ther. 25(2):379-391, 2017.

In a more particular aspect, these engineered vectors encode a functional USHER protein chosen from SANS, harmonin, whirlin, Myosin7A, Clarin-1 and Usherin, or a functional homologous or fragment thereof, and can be used to treat the USH1G, USH1C, USH2D, USH1B, USH3A, or USH2A syndrome respectively.

Of note, dual or multiple AAV recombinant vectors will be preferably used to bypass the AAV limited packaging capacity to 4.7 kb, which excludes some of the identified deafness genes such as MYO7A (USH1B), CDH23, PCDH15 (USH1F). In this case, the present invention may concern a vector system containing two or three therapeutic vectors, each encoding a part of the said proteins. Preferably, these vector systems contain at least two AAVs encoding at least half of said proteins.

The vectors of the invention may contain any feature (promoters, termination regions, etc) that favours the expression of the recombinant USHER protein inserted in their polynucleotide.

Promoters contemplated for use in the vector system of the invention include, but are not limited to, the natural promoter of each USHER gene, or an exogenous promoter such as cytomegalovirus (CMV) promoter, SV40 promoter, Rous sarcoma virus (RSV) promoter, chimeric CMV/chicken beta-actin promoter (CBA) and the truncated form of CBA (smCBA) (U.S. Pat. No. 8,298,818). In a specific embodiment, the promoter is a chimeric promoter comprising CMV and beta-actin promoter. Promoters can be incorporated into a vector using standard techniques known in the art. Multiple copies of promoters or multiple promoters can be used in the vector systems of the invention. In one embodiment, the promoter can be positioned about the same distance from the transcription start site as it is from the transcription start site in its natural genetic environment. Some variation in this distance is permitted without substantial decrease in promoter activity. A transcription start site is typically included in the vector.

Transcription termination regions can typically be obtained from the 3′ untranslated region of a eukaryotic or viral gene sequence. Transcription termination sequences can be positioned downstream of a coding sequence to provide for efficient termination. Signal peptide sequence is an amino terminal sequence that encodes information responsible for the relocation of an operably linked polypeptide to a wide range of post-translational cellular destinations, ranging from a specific organelle compartment to sites of protein action and the extracellular environment. Enhancers are cis-acting elements that increase gene transcription and can also be included in a vector. Enhancer elements are known in the art, and include, but are not limited to, the CaMV 35S enhancer element, cytomegalovirus (CMV) early promoter enhancer element, and the SV40 enhancer element.

DNA sequences which direct the polyadenylation of the mRNA encoded by the structural gene can also be included in a vector.

Methods for preparing viruses and virions comprising a heterologous polynucleotide or construct are known in the art. In the case of AAV, cells can be coinfected or transfected with adenovirus or polynucleotide constructs comprising adenovirus genes suitable for AAV helper function. Examples of materials and methods are described, for example, in U.S. Pat. Nos. 8,137,962 and 6,967,018.

The skilled person would easily determine if it is required, prior to the administration of the vector(s) of the invention, to enhance the permeability of the round window membrane as proposed in WO 2011/075838, depending on the target cell.

It was taught in a number of study that the therapeutic window for local gene transfer in mice affected by genetic deafness was embryonic or in early post-natal days (Ahmed et al, JARO 18:649-670 (2017); Zhang et al, Frontiers in Molecular Neuroscience, vol. 11, Art.221, 2018). Yet, the results obtained by the present inventors demonstrate that the gene therapy of the invention can be efficient not only when the vector is administered at these early stages of the mice life, but also far later, from P14 and until P30 and later, when the mice achieve adultlike hearing characteristics (Song L. et al, J Acoust Soc Am 119(4):2242-2257 (2006)).

At these late stages, the mice auditory system corresponds to the auditory system of an infant or adult human. Hence, the results obtained by the inventors suggest that the gene therapy used in the present invention can be efficient in humans after their birth, for example in infant patients that are diagnosed to suffer from a strong form of the USHER syndrome (USH1), or in adult patients that are diagnosed later, for example because they suffer from a milder form of the disease (USH2 or USH3).

Therefore, the vector of the invention, as defined above, will be particularly efficient when administered to human patients suffering from the USHER syndrome, in particular from the USHER 1 subtype, wherein said human patients have a developed and mature auditory system, especially the cochlea.

More preferably, the human patients treated by the vectors of the invention are new-born, toddlers, infants, teenagers or adult humans. These human patients are therefore not human embryos nor foetuses. In other words, the administration of the vectors of the invention is not intended to be performed in utero.

In a preferred embodiment, the patients targeted by the present invention are preferably new-born human babies, typically younger than 6 months old, or even younger than 3 months old, if the USHER syndrome is diagnosed that young. These human babies are more preferably between 3 months and 1 year. As a matter of fact, the earlier the gene therapy is performed, the better the outcomes will be.

It is also possible to administer the vectors of the invention, as defined above, to older human patients, such as toddlers (2-6 year old), infants (6-12 year old), teenagers (12-18 year old) or adult humans (18 years and over), as soon as the USHER syndrome is diagnosed.

Altogether, it is therefore preferred to administer the vectors of the invention to human patients aged between 3 months and 25 years. In another particular embodiment, the patients of the invention are human beings that are 6 years and older, i.e., the administration of the treatment occurs when their Central Nervous System is completely mature.

The vectors or the viral particles of the invention as described above can be incorporated into any pharmaceutical compositions that are suitable for an administration to a human being.

These pharmaceutical compositions comprise at least one vector of the invention and/or at least one viral particle containing said vector, and a pharmaceutically acceptable carrier.

In other words, the present invention relates to the use of the vectors of the invention or their viral particles, as described above, for manufacturing pharmaceutical compositions intended to prevent and/or treat patients having a mature auditory system, especially human beings suffering from the above-cited USHER disorders. More specifically, the present invention relates to the use of the vectors of the invention or their viral particles, as described above, for manufacturing pharmaceutical compositions intended to prevent and/or treat patients having a mature auditory system, especially human beings suffering from the USHER 1G disorder.

As used herein, the term “pharmaceutically acceptable carrier” refers to any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it can be preferable to include isotonic agents, for example, sugars, polyalcohols such as mannitol, sorbitol, or sodium chloride in the composition. Pharmaceutically acceptable carriers can further comprise minor amounts of auxiliary substances such as wetting or emulsifying agents, preservatives or buffers, which enhance the shelf life or effectiveness of the vector(s) and/or viral particle(s) or of the pharmaceutical compositions containing same. This definition applies to all aspects and embodiments of the present invention.

The pharmaceutical compositions of the invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The form used depends on the intended mode of administration and therapeutic application. Typical compositions are in the form of injectable or infusible solutions.

Pharmaceutical compositions typically must be sterile and stable under the conditions of manufacture and storage. The pharmaceutical composition of the invention is preferably formulated as a solution, microemulsion, dispersion, liposome, or other ordered structure suitable to high drug concentration. Sterile injectable solutions can be prepared by incorporating the vector(s) and/or the viral particle(s) of the invention in the required amount in an appropriate solvent optionally with one or a combination of ingredients enumerated above, as required, followed by filtered sterilization. Generally, dispersions are prepared by incorporating the vector(s) and/or the viral particle(s) of the invention into a sterile vehicle that contains a basic dispersion medium and optionally other ingredients from those enumerated above, as required. In the case of sterile lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be achieved by including an agent in the compositions that delays absorption, for example, monostearate salts and/or gelatine.

The pharmaceutical compositions of the invention include a “therapeutically effective amount” of the vectors of the invention and/or the viral particles containing same. A “therapeutically effective amount” refers to the amount of the vectors of the invention and/or the viral particles containing same, that is effective at dosages and for periods of time necessary, to achieve the desired therapeutic or preventive result in a subject in need thereof, in this case to efficiently treat hearing impairment, preferably the USHER syndrome, yet preferably USH1G, USH1B, USH1C, USH2D, USH2A, or USH3A syndrome, and even more preferably the USH1G disorder, without unacceptable toxicity or undesirable side effects.

A therapeutically effective amount of the vectors of the invention and/or the viral particles containing same can vary according to factors such as the disease state, age, sex, and weight of the subject, and the ability of said vectors and/or said viral particles to elicit a desired response in same. A therapeutically effective amount can also be one in which any toxic or detrimental effects of the vectors and/or the viral particles are outweighed by the therapeutically beneficial effects.

Dosage regimens can be adjusted to provide the optimum desired response (e.g., to cure or prevent the symptoms of the USH1G syndrome). For example, a single bolus can be administered, several divided doses can be administered over time or the dose can be proportionally reduced or increased as indicated by the exigencies of the therapeutic situation. It can be especially advantageous to formulate injectable compositions in dosage unit form for ease of administration and uniformity of dosage. Dosage unit form as used herein refers to physically discrete units suited as unitary dosages for the subjects to be treated; each unit containing a predetermined quantity of the vector(s) and/or the viral particle(s) of the invention calculated to produce the desired therapeutic effect in association with the required pharmaceutical carrier. The specification for the dosage unit forms can be dictated by and directly dependent on (a) the unique characteristics of the vector(s) and/or the viral particle(s) and the particular therapeutic effect to be achieved, and (b) the limitations inherent in the art of formulating such vector(s) and/or the viral particle(s) for treating Usher syndrome, in particular USH1 syndrome, preferably USH1G syndrome, in a subject in need thereof.

In some embodiments, the composition of the invention contains from 10⁶ to 10¹⁵ particles/mL or from 10¹⁰ to 10¹⁵ particles/mL, or any values there between for either range, such as for example, about 10⁶, 10⁷, 10⁸, 10⁹, 10¹⁰, 10¹¹, 10¹², 10¹³, 10¹⁴ or 10¹⁵ particles/mL. In one embodiment, the composition of the invention contains more than 10¹³ of AAV particles/mL.

The present invention also relates to preventing or treating methods involving the administration of the vectors of the invention and/or viral particles and/or pharmaceutical compositions containing same, to patients, especially human patients having a mature auditory system, suffering from the USHER syndrome, in particular USH1 syndrome, preferably USH1G syndrome. All the embodiments disclosed above apply to said preventing or treating methods.

In other words, the present invention relates to a method for preventing or treating the Usher syndrome in a human being having a mature auditory system, comprising administering to said human being, preferably by injection, the vectors of the invention and/or viral particles and/or pharmaceutical composition containing same. In a particular embodiment, said injection is a cochlear injection, as described below.

In another aspect, the present invention relates to the use of the vectors of the invention and/or the viral particles containing same for manufacturing pharmaceutical compositions intended to prevent or treat human beings having a mature auditory system and suffering from Usher syndrome, in particular from USH1 syndrome, preferably from USH1G syndrome.

The vector of the invention and/or a viral particle and/or a pharmaceutical composition containing same, can be administered both for preventing the loss of hearing or balance or of vision before it occurs, and for partially or totally restoring the hearing/balance/vision capacity when said loss has already occurred.

Multiple routes of delivery to the inner ear have been explored. These include injection into the perilymphatic spaces via the round window membrane (RWM) and via the oval window and injection into the scala tympani or scala vestibule via cochleostomy. Distribution throughout the perilymphatic spaces has been demonstrated for all these routes of delivery. Furthermore, it has been demonstrated that advection flow through the cochlea and vestibular organs can facilitate distribution of therapeutic agents from the site of injection to more distant regions of the inner ear. Delivery into the endolymphatic spaces has also been explored via cochleostomy into the scala media, via canalostomy and by injection into the endolymphatic sac. These approaches have also yielded broad distribution but face the added challenge of breaching the barrier between high potassium endolymph and perilymph. Disruption of the barrier poses two potential problems. First, leakage of high potassium into the perilymphatic spaces that bathe the basolateral surface of hair cells and neurons can chronically depolarize these cells and lead to cell death. Second, breach of the tight junctions between endolymph and perilymph can lead to rundown of the endocochlear potential which typically ranges between +80 and +120 mV. Rundown of the endocochlear potential reduces the driving force for sensory transduction in hair cells and therefore leads to reduced cochlear sensitivity and elevated auditory thresholds. Avoiding these complications is particularly challenging in the adult cochlea. However, by targeting endolymphatic spaces in the vestibular system, which does not have an endolymphatic potential, but are continuous with cochlear endolymph spaces, these confounding issues may be minimized while still providing sufficient distribution within the cochlea (Ahmed et al, JARO 18:649-670 (2017)).

The cochlea is highly compartmentalized and separated from the rest of the body by the blood-cochlear barrier (BCB), which minimizes the therapeutic injection volume and leakage into the body's general circulation system, to protect cochlear immune privilege and reduce the chance of systemic adverse immune responses. As the hair cells and supporting cells in the cochlea normally do not divide, the cells in the cochlea remain stable, therefore making it possible to use nonintegrating viral vectors (e.g., AAV) for sustained transgene expression.

The semi-circular approach has been suggested as a promising injection route for future cochlear gene therapy in human trials since the posterior semi-circular canal also appears to be accessible in humans (Suzuki et al., Sci. Rep. 7:45524 (2017); Yoshimura et al., Sci. Rep. 8:2980 (2018).

In the context of the invention, the typical mode of administration of the vectors of the invention and/or viral particles and/or pharmaceutical compositions containing same is a local administration, or intratympanic (in the middle ear) or intracochlear. This local administration can be unilateral or bilateral. In a preferred embodiment, the administration of the vector of the invention and/or viral particle and/or pharmaceutical composition containing same is bilateral. As a matter of fact, the results of the present inventors show that bilateral administration leads to better results.

In a more preferred embodiment, the vectors of the invention and/or viral particles and/or pharmaceutical compositions containing same are delivered to a specific location using stereostatic delivery, particularly through the tympanic membrane or mastoid into the middle ear of a subject. This administration can be unilateral or bilateral. In a preferred embodiment, the administration of the vector of the invention and/or viral particle and/or pharmaceutical composition containing same is bilateral.

More precisely, the vectors of the invention and/or viral particles and/or pharmaceutical compositions containing same can be administered by using a micro-catheter that will be carried out either through the oval window using laser stapedotomy (trans-stapes) or transmastoid/trans-round window (Dai C. et al, JARO, 18:601-617, 2017). This local administration can be unilateral or bilateral. In a preferred embodiment, the administration of the vector of the invention and/or viral particle and/or pharmaceutical composition containing same is bilateral.

In this mode of the invention, the USHER gene is administered to the subject by in vivo gene therapy, so that the gene product/protein of interest is produced in situ in the appropriate auditory cells.

### BRIEF DESCRIPTION OF THE DRAWINGS

The FIG. 1 shows the transduction profile after cochlear injection of Anc80-GFP and AAV8-GFP through the round window of mature wild-type mice (p20). Around 100% of IHC were transduced towards the cochlea for both Anc80 (A,B) and AAV8 (D,E). The vestibular organs were transduced with a high variability depending on the injection (G), with no significant difference between Anc80 (C) and AAV8 (F). (G) Quantification of the transduction rate depending on the localization and the hair cell type in the cochlea (n=7 for Anc80, stripped, n=4 for AAV8, grey) and in the vestibule (n=7 for Anc80 and n=4 for AAV8).

The FIG. 2 shows that Anc80-Sans and AAV8-Sans gene therapy at neonatal stages restores both vestibular and auditory function. After Anc80-GFP injections at P2, the vast majority of hair cells of the organ of Corti (A, B) and of the macula (C) was transduced. Also, after injection of AAV8-GFP, the majority of VHC (F) and IHC were transduced but OHC transduction was lower compared to Anc80 (D,E). (G) The transduction rate depends on the location of the hair cell within the cochlear spiral (n=8 for Anc80, stripped, n=7 for AAV8, gray) and was very high in the vestibule (n=3 for Anc80 and for AAV8). (H) Anc80-Sans and AAV8-Sans gene therapy partially restores the ABR thresholds with no significant difference between the two vectors. (1) Bar graph showing a complete restoration of vestibular function evaluated after gene therapy at P2 with Anc80-Sans and AAV8-Sans measured with the platform test performed at p30 with no significant difference of the mean time on platform with the wild type mice (p>0.05—Mann-Whitney test). Scale bars: 100 μm for A and D; 10 μm for B,C,E,F.

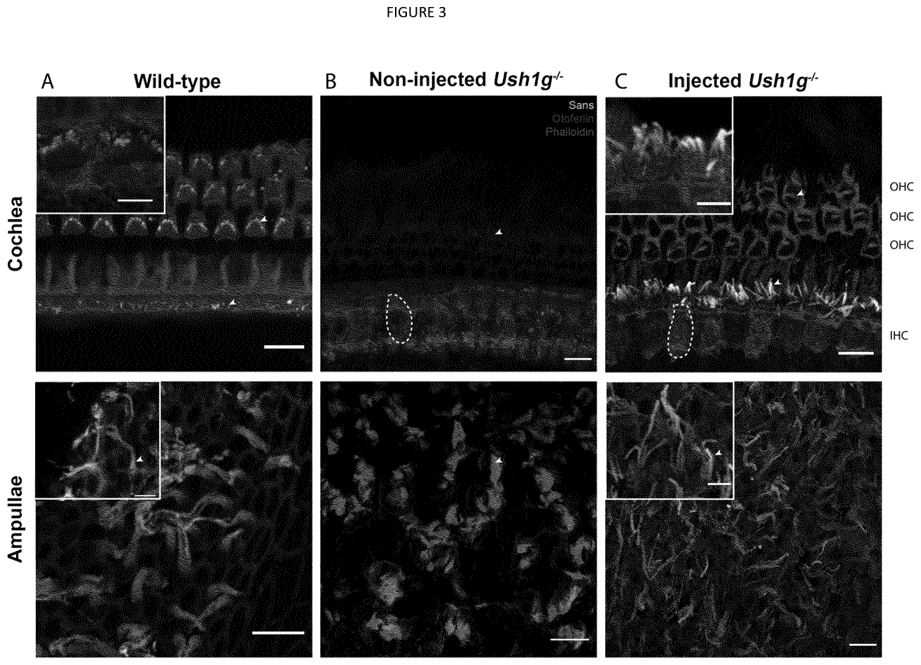

The FIG. 3 shows that the viral transfer of Sans cDNA at a mature stage restores sans expression and targeting in the IHC and VHC. Confocal images of hair cell immunolabelled for otoferlin (in red), for actin (in purple), and for sans (in green). (A) Sans is detected at the tip of stereocilia in the OHC, IHC, and VHC of the wild type mice at mature stage (P40). (B) Only no specific staining is seen in the inner hair cells of a P40 non-injected Ush1g^{-/-} mouse. (C) After injection at P14, sans expression and targeting are restored in the cochlea and the ampullae of a Ush1g^{-/-} mice in IHC and VHC, but not in the OHC. Scale bars: low magnification scans 10 μm, high magnification scans 5 μm.

The FIG. 4 shows the restoration of cochlear and auditory phenotype after Anc80-Sans delivery through the round window of Ush1g^{-/-} mice at mature stage. (A) Low and intermediate magnification scanning electron micrographs of organ of Corti of wild-type, non-injected Ush1g^{-/-}, and injected Ush1g^{-/-} mice, showing a partial restoration of hair bundle architecture of IHC and OHC (injection at P18; examination at P112). Scale bars: upper, 50 μm; lower 1 μm. (B) ABR thresholds in P35 wild-type (n=3), non-injected Ush1g^{-/-} (n=5), and injected Ush1g^{-/-} mice (n=12), showing a partial recovery on the 10, 15, and 20 kHz frequencies. (C) ABR traces, recorded in a wild-type mouse, a rescued Ush1g^{-/-} mouse (Ush1g^{-/-} injected) and an Ush1g^{-/-} mouse Ush1g^{-/-}, showing similar waveforms in the wild-type and rescued mice.

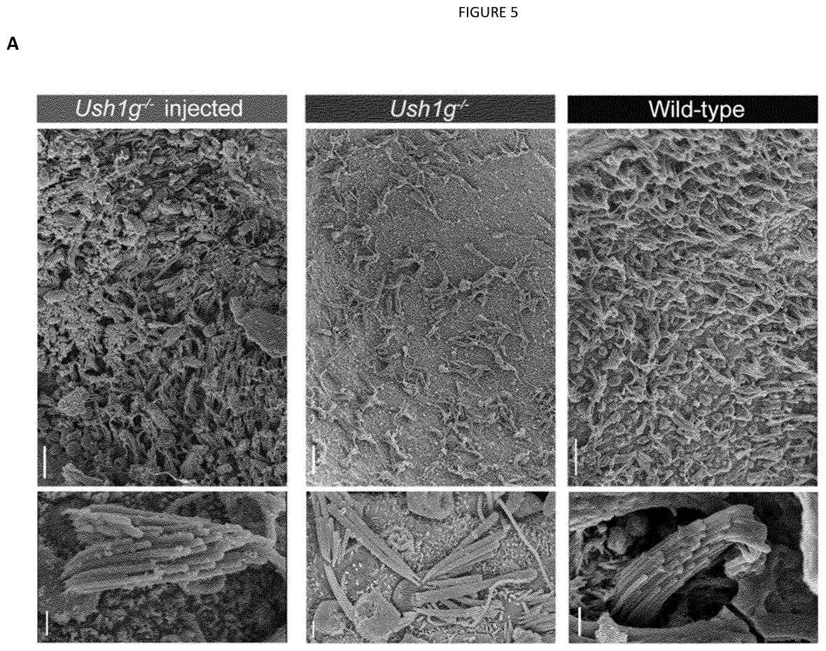

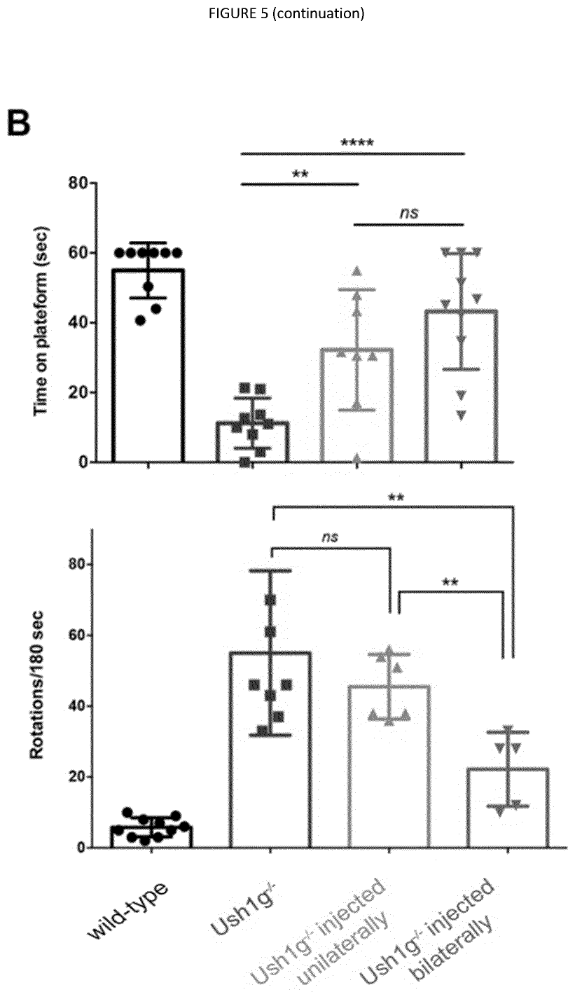

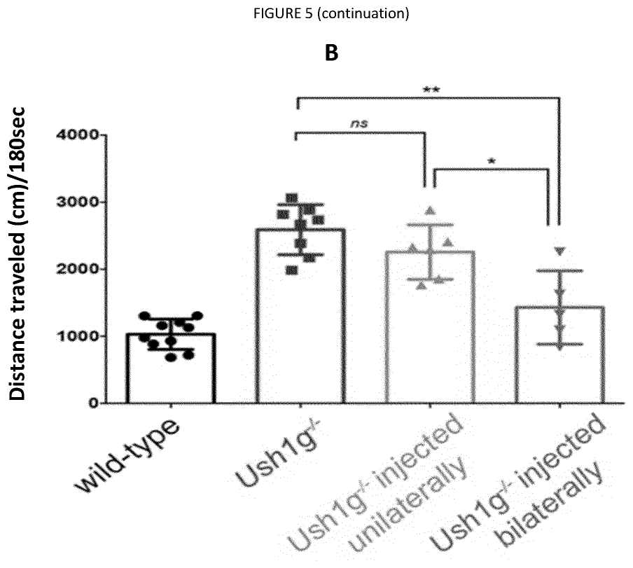

The FIG. 5 shows the vestibular function recovery in Ush1g^{-/-} mice after intracochlear Anc80-Sans delivery at a mature stage. (A) Electron microscopy analysis of utricular sensory epithelium of P112 wild-type, non-injected Ushg1g^{-/-} and injected Ush1g^{-/-} mice on P18 through the round-window membrane, showing that the gene delivery prevents vestibular hair cell degeneration, and restores the hair bundle architecture. (B) A significant improvement was found in the platform test (upper), with longer time spent on the platform after unilateral (p=0.004—Student's test) or bilateral (p<0.0001—Student's test) AAV-mediated gene therapy compared to untreated Ush1g^{-/-} mice. No significant difference found between the unilaterally- and bilaterally-injected mice (p=0.2—Student's test). Also, videotracking performed at P40 showed a significant improvement in the number of rotations in 180 seconds (middle, p=0.002, Mann-Withney test) the distance traveled after bilateral injection (lower, p=0.006, Mann-Withney test).

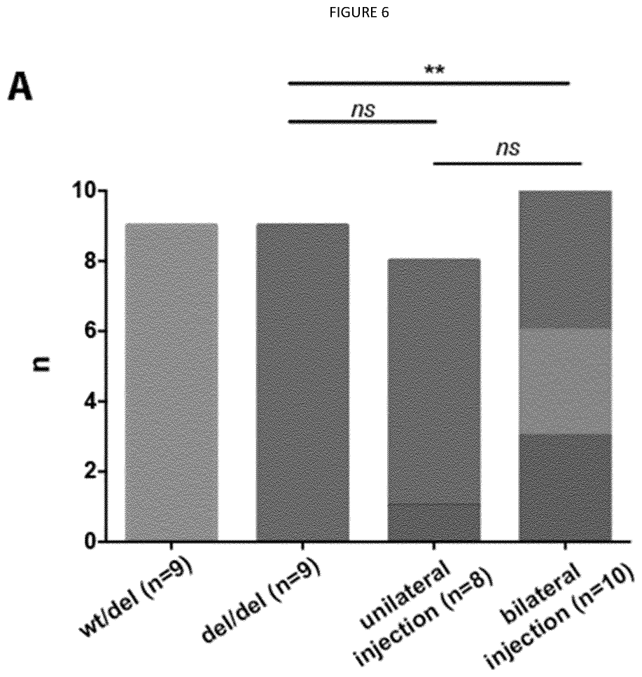

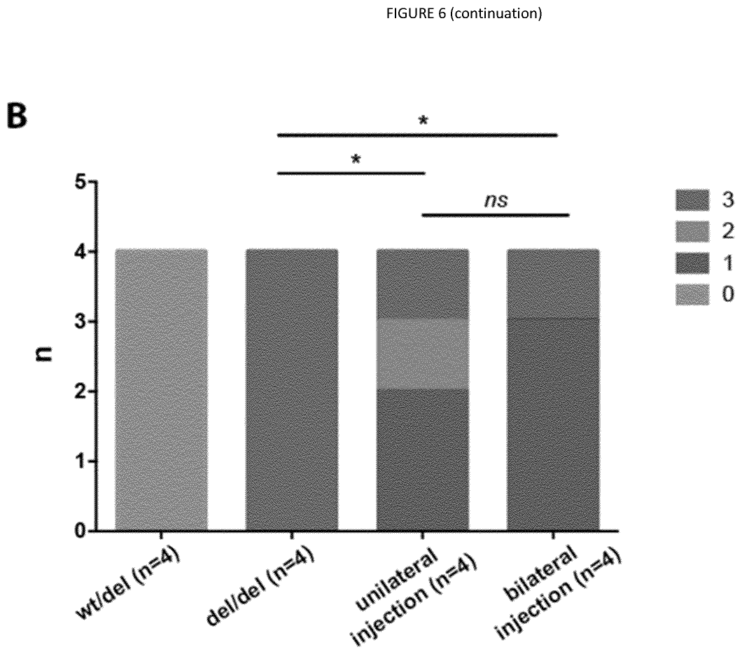

The FIG. 6 shows the vestibular testing with the swim test from 0 indicating normal swimming ability, and an increasing score toward 3 indicating increased swimming difficulty. (A) At P40, a significant difference was found between the scores obtained in the Ush1g^{-/-} mice injected bilaterally with the therapeutic vector (p=0.009—Chi2 test) and the untreated Ush1g^{-/-} mice but no difference was observed with those injected unilaterally (p=0.3—Chi2 test). (B) At p60, a significant improvement in swim scores in mice that received unilateral (p=0.04—Chi2 test) or bilateral injection (p=0.03—Chi2 test) was observed compared to non-injected Ush1g^{-/-} mice.

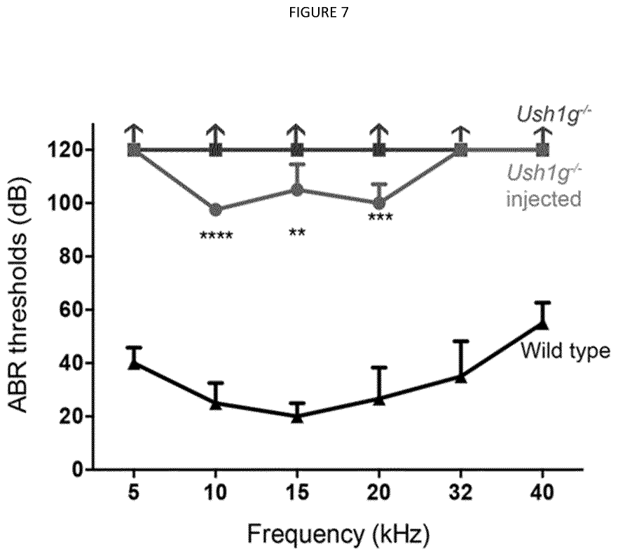

The FIG. 7 shows the hearing recovery on p60 after a single injection performed through the round window of mature Ush1g^{-/-} mice. Significant improvement of thresholds is observed at 10, 15, and 20 kHz for injected Ush1g^{-/-} mice (n=4) compared to uninjected Ush1g^{-/-} mice (n=6) (p=0.0001, ANOVA).

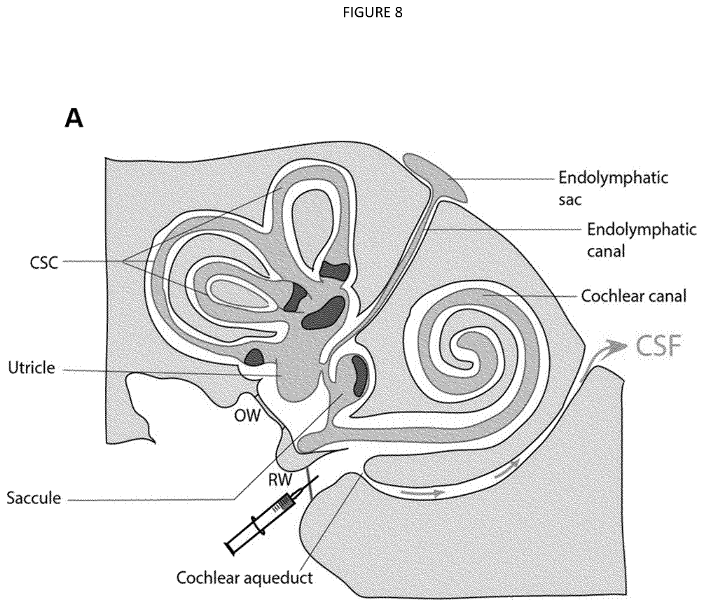

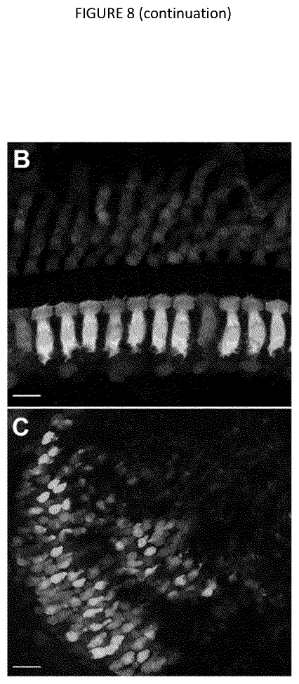

The FIG. 8 shows the transduction of the contralateral hair cells after intracochlear injection of Anc80-GFP through the round window (RW) membrane at neonatal stage. (A) After injection, the vector diffuses through the cochlear aqueduct that connects the perilymphatic space and the cerebro-spinal fluid (CSF), and goes to the contralateral ear through its own cochlear aqueduct. Organ of Corti (B) and utriculi macula (C) of the contralateral ear of an injected wild type mice at neonatal stage showing transduction of the vast majority of cochlear hair cells (GFP in green, Myosine VI in red). Scale bars: upper 10 μm, lower 20 μm.

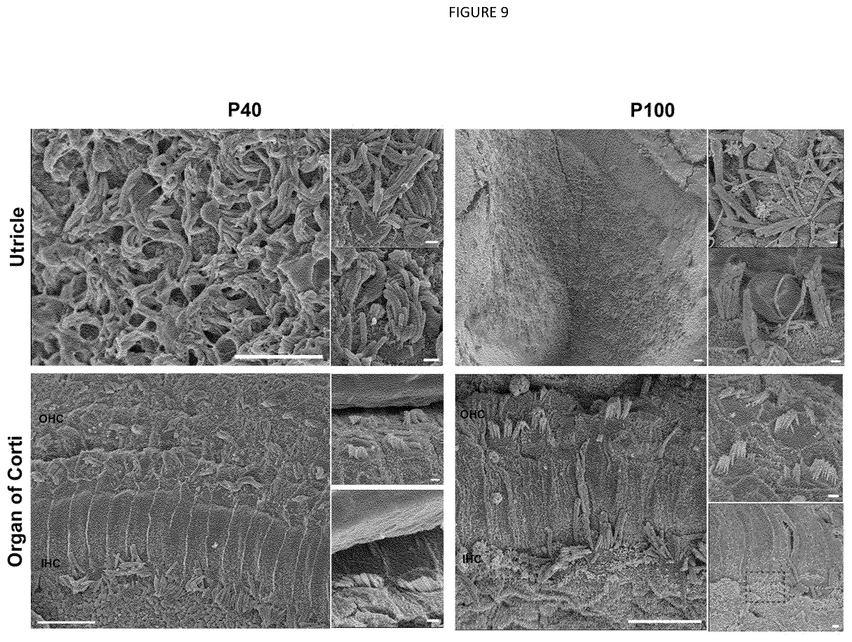

The FIG. 9 shows the morphological aspect of Ush1g^{-/-} hair bundle in utricle and cochlea at a mature stage. Low and intermediate magnification scanning electron micrographs showing the progressive degeneration of the hair bundle and the hair cells. At P40, the hair bundle is disorganized, and losses its normal staircase aspect in the utricle and in the cochlea. Also, the length of OHC and IHC stereocilia is significantly reduced (black arrow). At P100, a hair cell loss in both utricule and cochlea is observed. The low magnification of utricle shows an inhabited sensory epithelium, and a hair loss is also observed in the organ of corti (contoured in black). Finally, the VHC hair bundle displays either a complete disorganized aspect with large and long stereocilia, or a quite well-organized aspect but with a reduced length. Scale bars: low magnification micrographs 10 μm, intermediate magnification micrographs 1 μm.

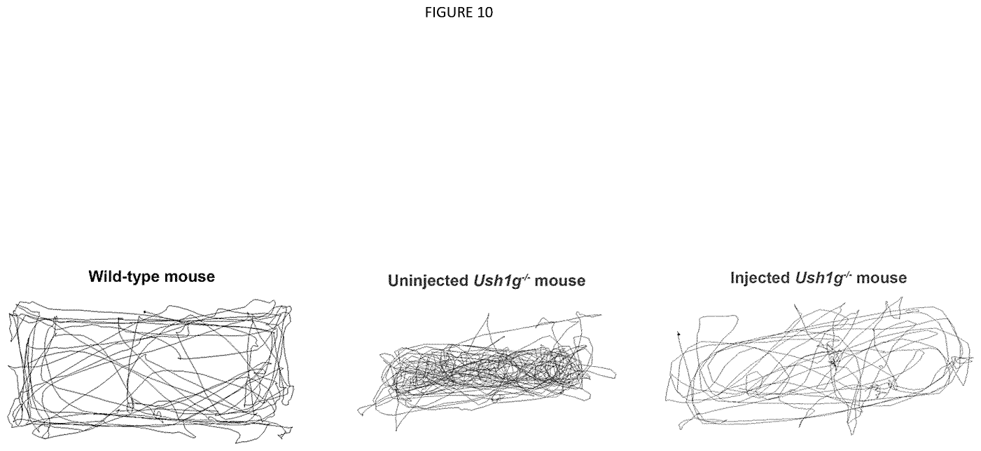

The FIG. 10 shows the recordings of the mouse displacements over a period of 3 minutes in a p40 wild-type mice, Ush1g^{-/-} mice and bilaterally injected Ush1g^{-/-} on p14.

### EXAMPLES

To identify efficient viral vectors that can be used in humans, the transduction efficiency of inner ear hair cells (IHCs) outer hair cells (OHCs) and vestibular hair cells (VHCs) was studied using several AAV vectors, including the Anc80L65 and AAV8 serotypes, at mature inner ear stages (P12-P30, between the 12th and 30th day of life). Recombinant vectors containing the green fluorescent protein (GFP) reporter gene driven by the ubiquitous promoter cytomegalovirus (CMV) were tested in wild-type mice. Viral preparations were microinjected directly into the cochlea of wild-type mice through the round window membrane (RWM) between P12 and P30. The cellular distribution of the GFP in the sensory epithelia of the cochlea and the vestibular end organs of the injected mice was evaluated by immunolabeling performed 1 to 2 weeks post-injection.

Since genetic forms of congenital inner ear defects, such as Ush1g, are typically diagnosed during the neonatal period, gene therapy approaches in animal models should therefore be tested at their adult stages, to determine to what extend it can reverse the inner ear defect.

The Ush1g^{-/-} mouse model, lacking the sans gene coding for the scaffold protein Sans, was used to investigate whether a local gene therapy in this mutant mouse could reverse the severe inner ear defect when administered to the mature hearing inner ear. To this end, a unilateral or bilateral delivery of the Anc80L65 driving the expression of the protein Sans was carried out through the round window membrane at mature stage between P12 and P30. Auditory brainstem responses and behavioral tests were used to evaluate the hearing restoration and vestibular function, respectively.

I. Material and Methods

Viral Vector Construct and Packaging.

To generate the p0101_CMV-SV40-Sans-bGH plasmid, the murine cDNA Sans sequence (GenBank accession no. NM_176847) was amplified by PCR (1401-pb amplicon, using a fwd sans 5′-GG GCG GCC GCC ACC ATG AATGACCAGTATCACCG-3′ primer (SEQ ID NO:14) and rev Sans 5′-GGAAGC TTA TCA TAGCTCCGTGTCCTCCA-3′ primer (SEQ ID NO:15)) from the pCMV-Tag 3B mSans plasmid (Snapgene) then digested by Notl and Hindlil and inserted into the pAAV.CMV.PI.EGFP.WPRE.bGH vector (Notl [971]/HindIII[1701], fragment of 4773pb, Addgene).

The resulting p0101_CMV-SV40-Sans-bGH plasmid was then packaged into the Anc80 capsid and produced by Penn VectorCore facility at a titer of 5.15×10¹² gc/mL.

Animals

Animal experiments were carried out in accordance with INSERM and Institut Pasteur welfare guidelines. Animals were housed in animal facilities accredited by the French Ministry of Agriculture for experiments on live mice.

For the generation and characterization of the Ush1g knockout mice, it can be referred to Caberlotto et al. (2011). Like Ush1G patients, Ush1g^{-/-} mutant mice displayed profound deafness (they showed no identifiable ABRs, even in response to sounds of intensities up to 110 dB sound pressure level) and vestibular dysfunction.

Genotyping of Ush1g^{-/-} recombinant animals was carried out by means of two PCR amplifications, using either oligo-fw1 (5′-GGCCTCGAAGAAGATCCTG-3′=SEQ ID NO:16) and oligo-rev (5′-GGCAAGTCAAAGGATCAGAT-3′=SEQ ID NO:17) to detect the wild-type allele (460-bp amplicon) or oligo-fw2 (5′-CAGTTTCCCCATGTTGATCACCAAC-3′=SEQ ID NO:17) and oligo-rev to detect the presence of a deleted allele lacking Ush1g exon 2 (332-bp amplicon) and wild-type (1964-bp amplicon) alleles. All studies were performed on mixed C57BL/6j genetic background.

Vector Delivery to the Cochlea

All surgical procedures and viral injections were carried out in a Biosafety level 2 Laboratory. Mice were anesthetized with isoflurane (4% for induction and 2% for maintenance). In order to reduce pain, mice received, at the beginning of the surgery, a subcutaneous injection of an analgesic, meloxicam (Metacamo, 0.2 mg/kg/day), and a subcutaneous injection of a local anesthesia in the retro-auricular region (Laocaïne®, 5 mg/kg). The anesthetized animal was placed on a thermopad throughout the procedure until the mouse was totally awake. The AAV vector was injected into the cochleae of 12- to 30-day-old Ush1g^{-/-} and wt mice.

Intracochlear injection was carried out as described by Akil et al. (2012). The left and/or right ear were approached via a retro-auricular incision. A small hole was made in the otic bulla with a 25G needle and expanded as necessary with forceps to access to the basal turn of the cochlea, and then widen sufficiently to visualize the stapedial artery and the round window membrane (RWM). The RWM was gently punctured in the center with a glass pipette and remained in place until efflux stabilized. The viral solution (2 μl of AAV Anc80-Sans at a titer of 5.15×10¹² gc/mL) was injected through the RWM using a pump system coupled to a glass micropipette (Sutter® P97). The RW niche was sealed quickly after pulling out the pipette with a small plug of muscle secured with a small drop of biological glue (Vetbondo 3M) placed on the muscle, to avoid leakage from the round window and with a small plug of fat to close the opening of the bulla.

Audiological Tests

ABRs to sound stimuli were recorded and analyzed as previously described (Emptoz et al., 2017). ABRs to sound stimuli were recorded at least 15 days post-injection. Briefly, the mice were anesthetized by an intraperitoneal injection of a mixture of ketamine hydrochloride (100 mg/kg) and xylazine hydrochloride 2% (10 mg/kg) and placed on a sound-proofed chamber. Subdermal needle electrodes were placed at the vertex, the ipsilateralmastoid (Emptoz et al. 2017), and on the back. Pure tone stimuli (bursts) were used at frequencies of 5, 10, 15, 20, 32 and 40 kHz. The hearing threshold was defined as the lowest stimulus level at which ABR peaks for waves I-V were clearly defined and repeatedly present upon visual inspection.

Behavioral Analysis

In order to determine vestibular deficits, different behavioral tests were performed (Hardisty-Hughes et al., 2010). The balance was evaluated with the platform test: The mouse was positioned on a small platform (7 cm×7 cm) at a height of 29 cm, and how many times the mouse fell off the platform over a period of 1 min was observed. The test was repeated three times and average time spent on the platform was collected during the monitoring period. The swimming ability of each mouse was also scored in a container (at least 15 cm deep) filled with water at 25-26° C. over a period of 1 min. Score of 0=Swims. Mouse body is elongated and the tail propels in a flagella-like motion; 1=Irregular swimming (vertical swimming, swimming in a circle, swimming on side, swimming in an unbalanced manner; 2=Immobile floating; 3=Underwater tumbling (the mouse must be rescued at once). Finally, the mice behavior was recorded and analyzed with the EthoVision XT video tracking software (Noldus Information Technology®, Wageningen, The Netherlands). The mouse was placed in its cage and tracked by video over a period of 3 minutes. The location and movement of the nose point, center point, and tail base of the animal were detected. The recorded parameters were as follows: the distance covered during the 3 minutes (in cm), the mean speed (cm/sec), the number of rotations in the clockwise and anticlockwise directions, and the time spent in the center of the cage.

II. Results

Transduction Profile of Inner Ear Hair Cells Depends on the Delivery Stage

The cell tropism and infectiousness of various viral serotypes were compared before and after hearing onset in mice. First were compared the ability of AAV8 (2.8×10¹³ gc/mL, Addgene) and Anc80 (1.05×10¹³ gc/mL, SignaGene) to transduce IHCs at a mature stage. The transduction efficiency was analyzed after a single viral intracochlear injection of each vector containing the green fluorescent protein (GFP) reporter gene associated with the CMV promoter. The injection was performed through the RWM in wild-type C57BL/6 mice at P20. The inner ear organs (organ of Corti, ampula, utricule, and saccule) were micro-dissected and immunolabeled for myosin VI to show the IHCs, and GFP. Both AAV8 and Anc80L65 targeted the cochlear IHC and the vestibule hair cells (FIG. 1A-F), with a mean transduction rate for IHCs of 97±5.9% [83-100] for Anc80L65 (n=7) and of 94±7.5% [83-100] for AAV8 (n=4) (p=0.2, t-test and Mann-Whitney) (FIG. 1G). As for OHCs, the transduction rate was very low for both Anc80L65 (13±23.7% [0-61], n=7) and AAV8 (12±21.1% [0-43.6], n=4), with no significant difference between the two capsids (p=0.9, Mann-Whitney).

In the balance organs, the transduction rate of the vestibular hair cells (VHCs) was similar for both serotypes regardless the injection stage. At neonatal stage, the transduction rate of VHCs was much higher compared to that of cochlear hair cells (OHC and IHC) for both Anc80 and AAV8, without significant difference between the two serotypes (p=0.4, Mann-Whitney test): respectively 99±1.4% [98-100] (n=3, FIG. 2C) and 92±7.5% [85-100] (n=3, FIG. 2F). In contrast, at mature stage, the transduction of the VHCs was lower, with a great variability for both Anc80 (43±29.6 [11-90], n=5, FIG. 1C) and AAV8 (19±38.8% [0-78], n=4, FIG. 1F). No difference was found between Anc80 and AAV8 at a mature stage (p=0.2, Mann-Whitney test). These results show that both AAV8 and Anc80 transduced the IHC and vestibular hair cells with the same efficiency, suggesting that they may improve the restoration of hearing.

Sans Gene Therapy at Neonatal Stage Prevents Deafness and Balance Disorders

It was previously found that there is a linear correlation between hearing threshold rescue and the percentage of transduced sensory cells within the inner ear. So the effect of the cochlear delivery of an Anc80 containing the murine cDNA sans with the CMV promoter (Anc80-CMV-Sans-WPRE, 5.15×10¹² gc/mL, Penn Vector Core) was tested at neonatal stage in Ush1g^{-/-} mice.

It was shown already that the intra-cochlear viral injection of an AAV8-Sans-internal ribosome entry site (IRES)-GFP virus at neonatal stage restored completely the vestibular function but only partially the auditory function (Emptoz et al., 2017). Anc80-CMV-Sans-WPRE injected through the round window membrane of P2 Ush1g^{-/-} mice cochlea did not significantly improve hearing rescue when compared to AAV8 (FIG. 2H, 1). The auditory function was partially restored, with no difference in hearing thresholds measured by auditory brainstem responses (ABR): the pure-tone average was 96±2.2 dB [87-114.5] and 90±3.1 respectively for Anc80 (n=11) and AAV8 (n=6) (p=0.1, unpaired t-test). This suggests that the number of transduced OHCs remains below that required to actually improve hearing rescue. Similarly, just like the AAV8, all vestibular tests (Hardisty-Hughes et al., 2010) indicate that Anc80-Sans delivery at P2 led to a complete restoration of the vestibular function (n=15). Unlike the untreated Ush1g^{-/-} mice, all treated Ush1g^{-/-} mice displayed no head tossing nor circling behavior; the trunk curl test, and the contact righting test were also normal; the platform test of treated Ush1g^{-/-} mice showed no significant difference with the wild-type mice (FIG. 2I), but difference was highly significant with the non-treated Ush1g^{-/-} mice (respectively 52±11.2 sec, n=15, and 8±9.8 sec, n=11, p<0.0001—Mann Whitney test). Also, all Ush1g^{-/-} injected mice showed a normal swimming behavior just like the wild type mice, whereas the non-treated Ush1g^{-/-} mice displayed underwater tumbling. Finally, the video-tracking showed that the injected Ush1g^{-/-} mice explored the field just like the wild type mice, and displayed significantly less rotations than non-injected Ush1g^{-/-} mice (respectively 5±2.1 and 52±23.8 rotations/180s, p=0.001—Mann-Whitney test).

Sans Delivery at a Mature Stage Restores Protein Expression and Localization

Since mice begin to hear around P12 of age, gene therapy at neonatal stages would be equivalent to in utero therapy in humans. Therefore, it is crucial to extend the therapeutic window to adult mice making genetic therapy possible to treat deaf patients several months after birth. The effect of a Sans gene therapy performed after the onset of auditory and balance functions, i.e., between P12 and P30, was then investigated. The Anc80-CMV-Sans-WPRE (5.15×10¹² gc/mL, Penn Vector Core) was injected through the round window membrane to Ush1g^{-/-} mice. First it was checked whether the Anc80-CMV-Sans-WPRE vectors were able to drive sans protein expression and localization in hair cells in vivo. The sensory epithelia of cochlea and vestibular organs was micro-dissected 4 weeks after injection, and immunolabelled for sans (FIG. 3). The fragmented stereocilia of IHC, OHC, and VHC in the sensory epithelia of the non-injected Ush1g^{-/-} mice were devoid of sans immunostaining. By contrast, in the injected cochlea, an obvious sans staining was observed at the tip of IHC and of VHC stereocilia, indicating that the virally driven protein was properly targeted. As expected from the transduction profile obtained with the Anc80-CMV-GFP at a mature stage, no expression of SANS was detected in the OHC (FIG. 1.A-C).

Sans Delivery at a Mature Stage Rescues Inner Hair Cells from Hair Bundle Defect and Degeneration

The structure of the hair bundle was analyzed by scanning electro-microscopy in Ush1g^{-/-} mice injected at a mature stage (between P14 and P29), and compared to those of non-injected Ush1g^{-/-} mice and Ush1g^{-/-} mice. In Ush1g^{-/-} mice, hair bundles of abnormal structure are readily visible as early as P2.5. The severity of hair bundle abnormalities gradually worsens with age, and by P22, the numbers and the length of stereocilia were dramatically reduced compared to the wild type mice in both IHCs and OHCs throughout the cochlea spiral, and the VHC stereocilia are collapsed (Caberlotto et al., 2011; Emptoz et al., 2017). After P22, a progressive degeneration of stereocilia in both cochlea and vestibular organs is observed, and a disorganization of the hair bundle with collapsed stereocilia that goes in a random direction is seen at P40. After P100, a loss of hair cells is observed, and stereocilia of VHC display a complete abnormal morphology with either a long collapsed large stereocilia, or a very short stereocilia (FIG. 9).

In the cochlea, the morphology is partially restored (FIG. 4), with more stereocilia in the higher row of OHC for injected Ush1g^{-/-} mice compared to non-injected Ush1g^{-/-} mice (24±3.6 vs 15±5.7 at P40, p=0.001, Mann-Whitney test). Also, the length of OHC stereocilia was increased after injection: 2.1±0.21 μm for injected Ush1g^{-/-} mice and 1.3±0.22 μm for non-injected Ush1g^{-/-} mice (p<0.0001, Student t-test). Finally, the number of sterocilia in the IHC was increased in the injected Ush1g^{-/-} mice compared to the non-injected mice, but the difference was not statistically significant. Morphological measures are presented in Table 1.

TABLE 1
Morphological measurement of stereocilia OHC, IHC and VHC analyzed using scanning
electro-micrographs. Inner ear hair cell stereocilia of wild-type mice, non-injected Ush1g⁻¹⁻
mice, and injected Ush1g⁻¹⁻ mice with Anc80-Sans were analyzed. Measures are: stereocilia
height of the higher row, number of stereocilia of one row (higher row for OHC and IHC, central
row for VHC), width of the larger stereocilia in the bundle for VHC. The number of stereocilia
was counted only for hair bundle with a relatively rescued shape.

| | | Non-injected | Injected Ush1g⁻¹⁻ | |
|---|---|---|---|---|
| | | Ush1g⁻¹⁻ mice | mice | Wild type mice |
| p40 | OHC | 1.7 ± 0.45 [0.95- | 1.7 ± 0.15 [1.52- | 1.9 ± 0.15 [1.69- |
| | stereocilia | 2.47] | 1.98] | 2.07] |
| | length (μm) | (n = 11) | (n = 6) | (n = 4) |
| | OHC- | 15 ± 5.7 [6-21] | 24 ± 3.6 [21-29] | 29 ± 5.4 [18-33] |
| | Number of | (n = 8) | (n = 6) | (n = 11) |
| | stereocilia | | | |
| | IHC | 3.3 ± 0.61 [2.63- | 3.6 ± 0.31 [3.13- | 3.4 ± 0.48 [2.95- |
| | stereocilia | 4.05] | 3.96] | 4.3] |
| | length (μm) | (n = 6) | (n = 7) | (n = 7) |
| | IHC- Number | 10 ± 2.7 [6-14] | 9 ± 0.9 [8-10] | 11 ± 0.9 [9-12] |
| | of stereocilia | (n = 8) | (n = 4) | (n = 7) |
| | VHC | 3.7 ± 1.39 [0.85- | 7.4 ± 0.54 [6.37- | 7 ± 1.11 [5.45- |
| | stereocilia | 6.37] | 8.41] | 8.40] |
| | length (μm) | (n = 16) | (n = 13) | (n = 8) |
| | VHC | 0.4 ± 0.15 [0.22- | 0.3 ± 0.05 [0.26- | 0.2 ± 0.01 [0.22- |
| | stereocilia | 0.73] | 0.39] | 0.24] |
| | width (μm) | (n = 16) | (n = 15) | (n = 2) |
| | VHC- Number | 6 ± 1.3 [4-9] | 6 ± 0.7 [5-7] | 6 ± 0.5 [5-7] |
| | of stereocilia | (n = 12) | (n = 10) | (n = 8) |
| >p100 | OHC | 1.3 ± 0.22 [1.1-1.7] | 2.1 ± 0.21 [1.72- | 1.8 ± 0.19 [1.6- |
| | stereocilia | (n = 8) | 2.4] | 2.2] |
| | length (um) | | (n = 9) | (n = 6) |
| | OHC- | 8 ± 1.4 [7-11] | 16 ± 1.39 [14-18] | 29 ± 3.9 [22-32] |
| | Number of | (n = 8) | (n = 10) | (n = 8) |
| | stereocilia | | | |
| | IHC | 3.9 ± 0.69 [3.2-4.8] | 3.3 ± 0.54 [2.8-4.1] | 3 ± 0.1 [2.9-3.2] |
| | stereocilia | (n = 4) | (n = 4) | (n = 4) |
| | length (um) | | | |
| | IHC- Number | 4 ± 0.9 [3-5] | 8 ± 0.6 [8-9] | 10 ± 0.9 [9-11] |
| | of stereocilia | (n = 4) | (n = 3) | (n = 4) |
| | VHC | 7.1 ± 2.43 [2.40- | 8.6 ± 1.30 [7.20- | |
| | stereocilia | 11.06] | 10.70] | |
| | length (um) | (n = 11) | (n = 9) | |
| | VHC | 0.6 ± 0.28 [0.28- | 0.3 ± 0.04 [0.25- | |
| | stereocilia | 1.24] | 0.36] | |
| | width (um) | (n = 13) | (n = 9) | |
| | VHC- Number | 5 ± 0.7 [5-6] | 5 ± 0.5 [5-6] | |
| | of stereocilia | (n = 2) | (n = 7) | |

Remarkably, viral Sans transfer to the mature cochlea of Ush1g^{-/-} mice through the round window prevented hair cell from degeneration and restored to near normal and typical staircase pattern of VHC stereocilia (FIG. 5). Furthermore, the morphology and the structure of the stereocilia are also restored: at P40, there was no difference in the stereocilia length of injected Ush1g^{-/-} mice when compared to that of wild-type mice (7.4±0.54 μm vs 7±1.11 μm respectively, p=0.4, Mann-Whitney test), whereas the non-injected Ush1g^{-/-} mice was significantly shortened (3.7±1.39 μm, p<0.0001, Student t-test). At P100, stereocilia are thinner compared to non-injected Ush1g^{-/-} mice (0.29±0.04 μm vs 0.6±0.28 μm, p=0.004, Student t-test).

Hearing Recovery after Sans Delivery at a Mature Stage

ABR recordings were analyzed for a total of 47 ears of injected Ush1g^{-/-} mice after the onset of hearing in mice (p12 to p30) (27 mice injected in the left ear only and 10 mice injected on both ears). A significant rescue of ABR thresholds was observed in 12 mice treated between p12 and p21 otherwise these mice would remain profoundly deaf (12/38 mice injected before P21, 32%) (FIG. 4). The improvement was significant for 10, 15, and 20 kHz compared to Ush1g^{-/-} mice on p35 (p<0.0001, ANOVA). This partial rescue was still present at p60 for the same frequencies (FIG. 7).

Recovery of the Vestibular Function

The vestibular phenotype was analyzed using a variety of behavioral tests carried out at least 15 days after the injection (Hardisty-Hughes et al., 2010).

The FIG. 5 shows the vestibular function recovery in Ush1g^{-/-} mice after intracochlear Anc80-Sans delivery at a mature stage. A significant improvement was found in the platform test (B, upper), with longer time spent on the platform after unilateral (p=0.004—Student's test) or bilateral (p<0.0001—Student's test) AAV-mediated gene therapy compared to untreated Ush1g^{-/-} mice. No significant difference found between the unilaterally- and bilaterally-injected mice (p=0.2—Student's test). Also, videotracking performed at P40 showed a significant improvement in the number of rotations in 180 seconds (B, middle, p=0.002, Mann-Withney test) the distance traveled after bilateral injection (B, lower, p=0.006, Mann-Withney test).

Contrary to the results obtained after a neonatal delivery through the round window, for which we found a total recovery of vestibular phenotype in all cases (FIG. 2), the unilateral delivery of Anc80-Sans through the round window at mature stage (p12-p30) led to a partial recovery of vestibular phenotype with a high variability between the injected Ush1g^{-/-} mice (FIG. 5). This variability was probably due to the variability of transduction rate in VHC (FIG. 1), and to the fact that at neonatal stage, the viral preparation could diffuse to the contralateral ear through the cochlear aqueduct and the cerebro-spinal fluid (FIG. 8), which is not the case in a mature stage.

Thus, the likely explanation for the partial rescue is that in the adult mice, there is a little to non-diffusion of the recombinant vector to the contralateral ear through the cochlear aqueduct and the cerebro-spinal.

In order to verify this hypothesis, bilateral injections were carried out and a variety of vestibular tests were performed. Interestingly, all behavioral tests indicate a significant improvement of vestibular function when compared the unilateral injection. In the balance platform test at P40, the time spent on the platform was much shorter in non-injected Ush1g^{-/-} mice (mean value 11±2.3 sec; n=9) than in mice injected unilaterally (32±6.1 sec; n=8; p=0.004—Student's test) or bilaterally (43±5.2 sec; n=10; p<0.0001—Student's test). Also, compared to non-injected Ush1g^{-/-} mice, the treated mice displayed significantly less head bobbing and circling after unilateral (respectively p=0.03 and p=0.03, Fisher test) and bilateral injection (respectively p=0.01 and p=0.01, Fisher test). Contact righting test was achieved to near wild type level for mice injected unilaterally (n=6/8) and bilaterally (n=8/10), whereas all non-injected Ush1g^{-/-} mice failed to reorient their body upon inversion of the tube in which they were placed (p=0.005 and p=0.002 respectively after unilateral and bilateral injection, Chi2 test). Likewise, whereas all non-injected Ush1g^{-/-} mice curled the trunk toward their tail when suspended by the tail, this behavior is less observed after unilateral (p=0.001, Chi2 test) and bilateral injection (p=0.002, Chi2 test).

The FIG. 6 shows the vestibular testing with the swim test from 0 indicating normal swimming ability, and an increasing score toward 3 indicating increased swimming difficulty. (A) At P40, a significant difference was found between the scores obtained in the Ush1g^{-/-} mice injected bilaterally with the therapeutic vector (p=0.009—Chi2 test) and the untreated Ush1g^{-/-} mice but no difference was observed with those injected unilaterally (p=0.3—Chi2 test). (B) At p60, a significant improvement in swim scores in mice that received unilateral (p=0.04—Chi2 test) or bilateral injection (p=0.03—Chi2 test) was observed compared to non-injected Ush1g^{-/-} mice.

While all the untreated Ush1g^{-/-} mice started drowning and had to be lifted out and rescued immediately, both unilaterally- and bilaterally-injected mice exhibited irregular swimming (12.5% and 30% of the unilaterally- and bilaterally-injected mice, respectively) or either stayed still floating (30% of the animals after bilateral injection).

Video-tracking performed at P40 showed that the circling behavior was significantly lower for bilaterally-injected mice compared to non-injected mice (respectively 22±10.4 vs 55±23.2 rotations in 180 seconds, p=0.002—Mann-Whitney test; FIG. 5). Also, the distance traveled in 180 seconds was lower for bilaterally-injected mice compared to non-injected mice (respectively 1429±547.4 vs 2589±372.9 cm, p=0.006—Mann-Whitney test). Finally, the injected mice travelled into the cage with a partially recovered behavior compared to the non-injected Ush1g^{-/-} mice, especially after a bilateral delivery of the cDNA Sans (FIG. 10). These data demonstrate that bilateral treatment improved the vestibular rescue and strongly suggest that the time window of gene therapy for vestibular deficit linked to user 1G syndrome is larger than initially suspected.

The present data strongly suggest that gene therapy carried out at adult stages can not only prevent the hair cell from degeneration, but also restores their structure and functions. In addition, these results significantly expend the therapeutic window for Ush1g^{-/-} mice and provide the first proof of concept that an equivalent clinical scenario is possible to treat patients with USH1G months after the initial damage.

### BIBLIOGRAPHIC REFERENCES

Adato, A., Michel, V., Kikkawa, Y., Reiners, J., Alagramam, K. N., Weil, D., Yonekawa, H., Wolfrum, U., El-Amraoui, A., & Petit, C. (2005). Interactions in the network of usher syndrome type 1 proteins. Human Molecular Genetics, 14(3), 347-356. https://doi.org/10.1093/hmg/ddiO3l
Ahmed, H., Shubina-Oleinik, O., & Holt, J. R. (2017). Emerging Gene Therapies for Genetic Hearing Loss. JARO—Journal of the Association for Research in Otolaryngology, 18(5), 649-670. https://doi.org/10.1007/s10162-017-0634-8
Asokan, A., Schaffer, D. V., & Samulski, R. J. (2012). The AAV vector toolkit: Poised at the clinical crossroads. Molecular Therapy, 20(4), 699-708. https://doi.org/10.1038/mt.2011.287
Bauß, K., Knapp, B., Jores, P., Roepman, R., Kremer, H., Wijk, E. V., Marker, T., & Wolfrum, U. (2014). Phosphorylation of the Usher syndrome 1G protein SANS controls Magi2-mediated endocytosis. Human Molecular Genetics, 23(15), 3923-3942. https://doi.org/10.1093/hmg/ddu104
Caberlotto, E., Michel, V., Foucher, I., Bahloul, A., Goodyear, R. J., Pepermans, E., Michalski, N., Perfettini, I., Alegria-Prévot, O., Chardenoux, S., Do Cruzeiro, M., Hardelin, J.-P., Richardson, G. P., Avan, P., Weil, D., & Petit, C. (2011). Usher type 1G protein sans is a critical component of the tip-link complex, a structure controlling actin polymerization in stereocilia. Proceedings of the National Academy of Sciences of the United States of America, 108(14), 5825-5830. https://doi.org/10.1073/pnas.1017114108
Dai, C., Lehar, M., Sun, D. Q., Rvt, L. S., Carey, J. P., MacLachlan, T., Brough, D., Staecker, H., Della Santina, A. M., Hullar, T. E., & Della Santina, C. C. (2017). Rhesus Cochlear and Vestibular Functions Are Preserved After Inner Ear Injection of Saline Volume Sufficient for Gene Therapy Delivery. JARO—Journal of the Association for Research in Otolaryngology, 18(4), 601-617. https://doi.org/10.1007/s0162-017-0628-6
Emptoz, A., Michel, V., Lelli, A., Akil, O., De Monvel, J. B., Lahlou, G., Meyer, A., Dupont, T., Nouaille, S., Ey, E., De Barros, F. F., Beraneck, M., Dulon, D., Hardelin, J.-P., Lustig, L., Avan, P., Petit, C., & Safieddine, S. (2017). Local gene therapy durably restores vestibular function in a mouse model of Usher syndrome type 1G. Proceedings of the National Academy of Sciences of the United States of America, 114(36). https://doi.org/10.1073/pnas.1708894114
György, B., Sage, C., Indzhykulian, A. A., Scheffer, D. I., Brisson, A. R., Tan, S., Wu, X., Volak, A., Mu, D., Tamvakologos, P. I., Li, Y., Fitzpatrick, Z., Ericsson, M., Breakefield, X. O., Corey, D. P., & Maguire, C. A. (2017). Rescue of Hearing by Gene Delivery to Inner-Ear Hair Cells Using Exosome-Associated AAV. Molecular Therapy, 25(2), 379-391. https://doi.org/10.1016/j.ymthe.2016.12.010
Hardisty-Hughes, R. E., Parker, A., & Brown, S. D. M. (2010). A hearing and vestibular phenotyping pipeline to identify mouse mutants with hearing impairment. Nature Protocols, 5(1), 177-190. https://doi.org/10.1038/nprot.2009.204
Hepper, P. G., & Shahidullah, B. S. (1994). Development of fetal hearing. Archives of Disease in Childhood, 71(3 SUPPL.), 81-87. https://doi.org/10.1136/fn.71.2.f81
Mustapha, M., Chouery, É., Torchard-Pagnez, D., Nouaille, S., Khrais, A., Sayegh, F. N., Mégarbané, A., Loiselet, J., Lathrop, M., Petit, C., & Weil, D. (2002). A novel locus for Usher syndrome type I, USH1G, maps to chromosome 17q24-25. Human Genetics, 110(4), 348-350. https://doi.org/10.1007/s00439-002-0690-x
Petrs-Silva, H., Dinculescu, A., Li, Q., Deng, W. T., Pang, J. J., Min, S. H., Chiodo, V., Neeley, A. W., Govindasamy, L., Bennett, A., Agbandje-Mckenna, M., Zhong, L., Li, B., Jayandharan, G. R., Srivastava, A., Lewin, A. S., & Hauswirth, W. W. (2011). Novel properties of tyrosine-mutant AAV2 vectors in the mouse retina. Molecular Therapy, 19(2), 293-301. https://doi.org/10.1038/mt.2010.234
Shnerson, A., & Willott, J. (1980). Ontogeny of the acoustic startle response in C57BL/6J mouse pups. J Comp Physiol Psychol, 94(1), 36-40.
Song, L., McGee, J., & Walsh, E. J. (2006). Frequency- and level-dependent changes in auditory brainstem responses (ABRs) in developing mice. The Journal of the Acoustical Society of America, 119(4), 2242-2257. https://doi.org/10.1121/1.2180533
Sorusch, N., Yildirim, A., Knapp, B., Janson, J., Fleck, W., Scharf, C., & Wolfrum, U. (2019). SANS (USH1G) Molecularly Links the Human Usher Syndrome Protein Network to the Intraflagellar Transport Module by Direct Binding to IFT-B Proteins. Frontiers in Cell and Developmental Biology, 7(October), 1-13. https://doi.org/10.3389/fcell.2019.00216
Suzuki, J., Hashimoto, K., Xiao, R., Vandenberghe, L. H., & Liberman, M. C. (2017). Cochlear gene therapy with ancestral AAV in adult mice: Complete transduction of inner hair cells without cochlear dysfunction. Scientific Reports, 7(March), 1-12. https://doi.org/10.1038/srep45524
Weil, D., El-Amraoui, A., Masmoudi, S., Mustapha, M., Kikkawa, Y., Lainé, S., Delmaghani, S., Adato, A., Nadifi, S., Zina, Z. Ben, Hamel, C., Gal, A., Ayadi, H., Yonekawa, H., & Petit, C. (2003). Usher syndrome type I G (USH1G) is caused by mutations in the gene encoding SANS, a protein that associates with the USH1C protein, harmonin. Human Molecular Genetics, 12(5), 463-471. https://doi.org/10.1093/hmg/ddg051
Yoshimura, H., Shibata, S. B., Ranum, P. T., & Smith, R. J. H. (2018). Enhanced viral-mediated cochlear gene delivery in adult mice by combining canal fenestration with round window membrane inoculation. Scientific Reports, 8(1), 2980. https://doi.org/10.1038/s41598-018-21233-z
Zhang, W., Kim, S. M., Wang, W., Cai, C., Feng, Y., Kong, W., & Lin, X. (2018). Cochlear Gene Therapy for Sensorineural Hearing Loss: Current Status and Major Remaining Hurdles for Translational Success. Frontiers in Molecular Neuroscience, 11(June). https://doi.org/10.3389/fnmo.2018.00221

1-14. (canceled)

15. A method for treating the USH1G syndrome, said method comprising administering to a human patient suffering from the USH1G syndrome, and having a developed and mature auditory system, a vector expressing the SANS protein encoded by the USH1 G gene, or expressing a functional homologous or fragment thereof.

16. The method of claim 15, wherein said homologous sequence or fragment shares at least 70% homology with the SANS protein.

17. The method of claim 15, wherein said SANS protein has the sequence reflected in SEQ ID NO:1 or in SEQ ID NO:3.

18. The method of claim 15, wherein said vector is a viral vector.

19. The method of claim 15, wherein said vector is an AAV vector.

20. The method of claim 15, wherein said vector is chosen in the group consisting of: AAV1, AAV2, AAV8, AAV9, and Anc80.

21. The method of claim 15, wherein said vector is an AAV2/8 vector or a Anc80 vector.

22. The method of claim 15, wherein said human patients are new born babies, toddlers, infants, teenagers or adults.

23. The method of claim 15, wherein said vector is administered bilaterally in said patients.

24. The method of claim 15, wherein the vector is contained within a pharmaceutical composition further comprising a pharmaceutically acceptable vehicle.

25. The method of claim 24, wherein said composition comprises at least one AAV particle comprising a polynucleotide encoding the SANS protein or a functional homologous or fragment thereof.

26. The method of claim 25, wherein said AAV particle is an AAV2/8 vector or a Anc80 vector.

27. The method of claim 24, wherein said composition comprises a vector containing the USH1G gene of SEQ ID NO:2 or of SEQ ID NO:19.

28. The method of claim 24, wherein said composition is injectable.

29. The method of claim 24, wherein said composition is administered to new born babies, toddlers, infants, teenagers or adults.

30. The method of claim 24, wherein said composition is administered bilaterally in said patients.
